(19) United States
(12) Patent Application Publication
(10) Pub. No.: US 2025/0259753 A1
(43) Pub. Date: Aug. 14, 2025

(54) Sharing of Healthcare Information Through a Dynamic Healthcare Database and Tokenization of Medical Data

(71) Applicant: Sanus Galt Inc., Princeton Junction, NJ (US)

(72) Inventor: Max Narovlyansky, Princeton Junction, NJ (US)

(21) Appl. No.: 19/054,001

(22) Filed: Feb. 14, 2025

Related U.S. Application Data
(60) Provisional application No. 63/553,594, filed on Feb. 14, 2024.

(51) Int. Cl. G16H 50/50; G16H 10/60; G16H 20/10

(52) U.S. Cl. CPC G16H 50/50; G16H 10/60; G16H 20/10

(57) ABSTRACT

A method for sharing medical data by creating a Medical Data Ocean (MDO) and a system for maintaining and using the MDO may include receiving medical data from disparate sources; performing datum distillation on the received medical data and integrating the distilled medical data to form integrated and distilled medical data; storing the integrated and distilled medical data into a medical database; receiving a request for information from the large-scale analytics of the integrated and distilled medical data; analyzing the integrated and distilled medical data to identify biological predictors and guide precision medicine; using the analysis of the integrated and distilled medical data to develop personalized treatment plans, predictive models, and/or providing a response to the request for information; and tracking the source of individual medical data from the integrated and distilled medical data used to determine the response to the requested information and/or to develop personalized treatment plans.

### BACKGROUND

Healthcare data is currently shared very sparingly. Furthermore, the existing structures do not provide any motivation for adoption of innovation. While the United States has many commercial examples of applications of Electronic Healthcare Records (EHRs), the absence of universal coverage and connectivity between silos of data results in a lack of large datasets and actionability.

Despite the abundance of medical data generated daily, the current healthcare environment lacks efficient methods for clinicians to obtain, process, and maintain a patient's complete medical history. This limitation not only impedes the delivery of personalized and effective care but also hinders the advancement of precision medicine, which relies heavily on the availability of comprehensive biomarker information and the ability to leverage shared experiences across patient populations. Furthermore, the identification and aggregation of rare disease patients remain a significant challenge, hampering the development of targeted pharmaceuticals and treatment plans.

If the pandemic has taught us anything it is the importance of health monitoring, from diagnostics that help to contain a potential outbreak or epidemic, to efficacy of therapy and association of risk factors with success. COVID vaccines have been developed and administered in record time of about nine months compared with the typical ten to fifteen years that includes extensive follow up. The initial emergency authorization did not envisage proper follow-up for what is in effect the largest clinical trial ever conducted. Neither the producers of vaccines nor the regulators at the Food and Drug Administration (FDA) or Center for Disease Control (CDC) have incentives to systematically examine the efficacy and adverse effects data. What is needed is an independent non-government entity that does not economically benefit from selling any healthcare product. Such a system can be used to build the trust of the public.

There is a substantial impediment to data sharing in the healthcare space. Specifically, the Health Insurance Portability and Accountability Act of 1996 (HIPAA) has imposed substantial privacy standards and restrictions on the sharing of personally identifiable information. HIPAA sets forth policies to protect all individually identifiable health information that is held or transmitted. Protected information can be used to identify, contact, or locate a single person or can be used with other sources to identify a single individual. When personally identifiable information is used in conjunction with one's physical or mental health or condition, health care, or one's payment for that health care, it becomes Protected Health Information (PHI).

Unfortunately, there are other impediments to patient data sharing as well. The patient data is held by the doctor in their respective EHR or the company conducting clinical or drug tests. Results, patient responses, and other effects on patients that may reside in the medical information of a patient are generally considered proprietary information to the company. Therefore, there is no motivation for connectivity or sharing of data by the entities holding the information, while those most likely to benefit do not have the access or connectivity to provide that information in a meaningful or efficient way.

Several deficiencies exist that prevent the formation of a large-scale medical information database(s) as shown and described herein. The origin of the problems lies in ownership and usage of medical data, which is currently overwhelmingly stored and used by institutions and not patients. This results in several inefficiencies affecting data fragmentation, availability, completeness, ownership and incentives.

The first on the list is data fragmentation, by which we mean the medical records exist in isolated pools, such as records of a single doctor, or a single hospital, or at most a hospital system that control multiple hospitals in a state or geographic location. Second, the business-to-business (B2B) focus of Electronic Health Records (EHRs) means there is limited data availability for the patient. While the patient has the right to the information providers have the right to the records that in practice means the industry, such as hospitals or pharmaceutical companies de facto “own” the data, which they store and use for their benefit not for the patient. Third, and this affects both the patient and potential industry partners is the completeness of records. While the hospitals are prohibited from selling patient's medical data without their consent, pharmaceutical companies carrying out clinical trials “own” the medical data acquired but have little or no access to the patient's records before or after the clinical trial. This important contextual information can go a long way towards explaining the efficiency of therapy in some patients, but not others, provided that enough patients are analyzed with sufficient diagnostic information to create an algorithm that explains biological measurements or biomarkers and predicts response in patients that fit certain biomarker patterns that correspond to various biological profiles.

This principle of data accrual has historical roots, for example, tribal shamans have accumulated and passed on expertise regarding medical herbs. The learning process was slow and inefficient. Remarkably, this inefficiency persists, not for lack of experimentation, but due to lack of systematic recording, sharing and hence learning and practically benefiting from the majority of medical practice. While there are nearly one billion doctor's visits in the US per year, only 25 thousand patients contributed to clinical trials. This means that only one patient's data is collected (in clinical trial format) per 40,000 doctor's visits. This ratio suggests vast potential for improvement in efficiency by systematically recording and reusing medical experience.

The central role played by pharmaceutical companies in drug development results in a natural repository of value in drugs, rather than patients. This sub-optimal situation is a consequence of regulatory structure, where once a drug is approved, any patient who receives it must pay the full price, regardless of performance. This disconnect has naturally led to financial pressure to oppose Outcomes-Based Medicine (OBM), where drug reimbursement is commensurate with performance or effect on the patient. OBM has the potential not only to deliver better results for the patient, but also greater financial rewards for drug development by rewarding superior medical outcomes. This can be particularly effective for many drugs that address markets with few patients that were created following the US Orphan Drug Act of 1983. The problem with existing metrics of medical efficacy is two-fold. First and foremost, efficacy is demonstrated statistically when a drug is approved and then administered to an individual patient with full compensation regardless of efficacy. Second, only a modest fraction of therapies administered are accompanied by using biomarkers achieving or even attempting Precision or Personalized Medicine (PPM). In most cases, doctors administer “Standard-Of-Care” (SOC) therapy, which from the perspective of the patient is a one-size-fits-all medicine delivered by drug maker with no financial stake in the efficacy for the individual. This dichotomy is particularly poignant in oncology, where therapy can cost several hundred USD while the potential biomarkers are neither developed as companion diagnostics for the therapy during the clinical trial nor accrued for refinement of therapy upon subsequent application. It is worth noting that new drugs get approved based on results on a few thousand patients cumulatively in all three phases of clinical trials, while the application for potentially millions of follow-on patients is typically left without record or refinement. After successful Phase III pivotal trials there are some Postmarketing Surveillance (PMS) requirements, but there is little incentives for drug developers to continue to refine application of their therapy resulting in very few instances of Phase IV clinical trials that accrue additional information. In summary, the existing B2B focus of data ownership impedes the creation of a large and interconnected data repository or network and hence a major challenge we address is lack of focus on the patient. While it is possible and desirable to obtain data from industry, it is by empowering the patient (B2C) that we create better outcomes by accruing a large a fundamentally egalitarian mega-database that we call the MDO.

Lack of sharing of data results in minimalist datasets with low predictive power or outright failure. Up to 40% of clinical trials fail due to inability to enroll enough patients, many after years of attempts to systematically apply a protocol. Lack of recruitment means an inability to make any use of the resulting information, which is grossly underpowered statistically. Ironically, this situation is quite common. It is even possible to find clinical trials with similar structures and lack of enrollment that could have combined their datasets eventually achieving statistically significant predictive power for whether a specific therapy fits a patient. Companies are even trying to repurchase data from failed clinical trials, but without the full contextual information about the patient this data has very limited utility and may at best allow efficacy of therapy to be segmented by gender, age, or race—the most basic and limited biomarkers.

Examples of various systems for bio-computation, especially micro-physiological systems (MPS) and bio-banking, including tools as well as associated functional models of disease, such as fibrosis or oncogenesis, are described in, for example, patent publications and registrations: US20170101615; U.S. Pat. No. 11,453,848; US20220193660; US20210095235; US20200386742; US20200239843; US20190345431; US20230029554; US20230272489; U.S. Pat. No. 11,723,347; U.S. Pat. No. 11,686,731; U.S. Pat. No. 11,629,385; U.S. Pat. No. 11,555,180; WO1998013783; and US20160135447.

Examples of processes and systems for de-identifying medical data can be found in, for example, US20170091391; US20200285771; U.S. Pat. No. 6,081,786.

Examples of cryptocurrencies including blockchain or Hyperledger (open ledger) technology that support cryptocurrencies it is built upon are disclosed in, for example, patent publications and registration: US20190095910; US20160292672.

Examples of electronic records management (EMR) or Electronic Health Records (EHRs) are disclosed in, for example, U.S. Pat. No. 5,924,074; US20180165416.

Existing medical data is fragmented across disparate sources, hindering the ability to perform comprehensive analytics and extract meaningful insights from the available data.

Traditional data storage solutions lack the ability to address the unique requirements of medical data, such as stringent privacy and security measures, as well as the ability to integrate diverse data types and formats.

The healthcare system lacks efficient methods for clinicians to obtain, process, and maintain a patient's complete medical history, impeding the delivery of personalized and effective care.

The identification and aggregation of rare disease patients remain a significant challenge, hampering the development of targeted pharmaceuticals and treatment plans.

The rapid obsolescence of medical information and the knowledge gap among physicians regarding the latest advancements in disease diagnosis and treatment can lead to misdiagnoses and compromised patient outcomes.

### SUMMARY

Exemplary embodiments of the systems and methods described herein may be used for sharing medical data in a meaningful and efficient way. Exemplary embodiments described herein may include the infrastructure to permit data sharing as well as incentive to encourage the adoption of information sharing. The incentives to adopt patient sharing may result in improved and/or dynamic data sets that may be used to deliver better health outcomes and create new avenues for engagement with patients for their data.

Exemplary systems and methods described herein may be used to de-identify patient and/or clinical data to permit sharing that is compliant with current regulations.

Exemplary embodiments of the systems and methods described herein may be used to create a database of medical data that may be interconnected and continuously updated to create living data records from patients. This database of medical data can be used for personalized patient care, clinical trials, medical development (drugs and other areas).

Exemplary embodiments of the systems and methods described herein relate to the healthcare data management field, specifically to a method for integrating and monetizing medical data from disparate sources to enable precision medicine.

There are issues in the existing technology, such as fragmented medical data across disparate sources, lack of efficient methods for clinicians to obtain and maintain complete patient medical histories, challenges in identifying and aggregating rare disease patients, and knowledge gaps among physicians regarding the latest advancements in disease diagnosis and treatment. Therefore, in order to solve the above problems, the present invention provides a comprehensive and integrated platform to consolidate medical data from various sources, while maintaining privacy and enabling monetization of the data for precision medicine applications.

### DESCRIPTION

The following detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention. It should be understood that the drawings are diagrammatic and schematic representations of exemplary embodiments of the invention that are not limiting of the present invention nor are they necessarily drawn to scale.

The present disclosure is generally related to storage and utilization of healthcare data. Embodiments described herein may be used to privatize healthcare data so that patients retain ownership of their own data, which then permits the exchange of data with the proper incentives.

The present disclosure provides a system for dynamic construction of one or more massive health-care databases centered around the patient(s). Exemplary embodiments described herein may be used to secure the active participation of patients by empowering them with precision medicine information and revenue sharing, growing the database. Exemplary embodiments also permit use of this healthcare database by third parties, such as doctors and pharmaceutical companies. Patients are encouraged to contribute their own patient information to the database based on improved medical healthcare in the form of personalized patient care as well as financial incentives by sharing in the revenues generated using their data.

Although embodiments of the invention may be described and illustrated herein in terms of systems and methods for facilitating the sharing of medical data, embodiments of this invention are not so limited but are additionally applicable to other data types that may include intrinsic value or for which sharing is desirable. Furthermore, although embodiments of the invention may be described and illustrated herein in terms of combinations of data share and revenue sharing, embodiments of the invention are not intertwined. Instead, the foundation of underpinning a digital currency to a set value or valuable digital assets, such as information, has its own applications. Accordingly, exemplary embodiments of the system and methods described herein may be used in various combinations and are all within the scope of the instant disclosure.

For use by either the patient or third parties, the medical information received into the system described herein from various patients is stripped of all personal identification.

Substantial regulatory systems protect user information, especially as it applies to health information. Essentially, information that can be used to identify, contact, or locate a single person or can be used with other sources to identify a single individual is generally protected (herein referred to as “Protected Information” or “PI”) and has substantial limitations on sharing such information. Two of the most important identifiers are photographic images that include the face or any other characteristic that could uniquely identify the individual, or primary personal identifiable information (PII). The latter can include any primary patient health information (PHI). Accordingly, exemplary embodiments of the systems and methods described herein eliminate and remove protected information before it reaches the database, so that this information cannot be shared with other patients or industry.

We consider all uses of biomedical data mentioned herein examples of bio-computation. Regardless of specific application in personalized medicine, drug development, or fundamental research, the systems and methods described herein permit inputs and outputs of information.

However, if all patient information is added to the system, then the system may become too large and may require substantial resources and infrastructure, but which may not have efficient or useful associations for use in the desired bio-computations. Therefore, to be actionable (suitable for bio-computation), exemplary embodiments of the systems and methods described herein may include sub-systems and sub-methods for distilling, filtering, or otherwise analyzing the datasets before the data is added to the database.

In an exemplary embodiment, to be actionable, the input information is filtered to retrieve data with a high physiological relevance. Determining the rank (e.g. low, high) of physiological relevance is relative and can be determined based on the system requirements. For example, the threshold to be “high” physiological relevance may be increased if the desirable database size is limited so that only the most relevant and useful data is added to the database. However, if additional infrastructure is available for the database, the “high” physiological relevance may be based on the actual contributions of the associations to the overall database and the desired bio- computations. Therefore, data that has no associations for bio-computation may have low or minimal physiological relevance. If the data has more associations for use in bio-computations, then the data's physiological relevance increases. The threshold to determine if the physiological relevance is “high” depends on the type of data and the available associations within a dataset, and in the diversity of the data that is available into the system. For example, if normal datasets from a patient comes with five associations, (e.g. ethnicity, age, marker, treatment, response), but a dataset comes in with four associations (e.g., does not have the response information), but the data is for an ethnic category in which data is lacking, then the dataset relevance may be increased, although the number of associations is actually less than other datasets. The physiological relevance may therefore depend on the time of the dataset, the quality and/or quantity of associations that are available or made within the dataset, the rarity or scarcity of one or more of the data attributes of the dataset compared to the datasets coming into the system, the detail of the dataset, the biomarkers, among other considerations. In an exemplary embodiment, the most valuable data can be found in patient cases where precision measurements of biological entities of high relevance or biomarkers are found together with patient response to therapy.

After filtering and compilation of the cleaned (including non-personal identifiable information) data, the large-scale database can be created. This large-scale database may be used to determine patterns of biomarkers to be correlated with response. The information contained in the database can benefit a patient directly, for example by helping them to narrow down the identification for pathology based on symptoms described by other users, or it helps to access the right information to arrive at a differential diagnosis. The database can be used to facilitate drug development, the application of precision medicine, or to carry out basic research. Because the access to the database is on commercial basis, we consider all these institutional users as “industry”, to distinguish them from individual patients.

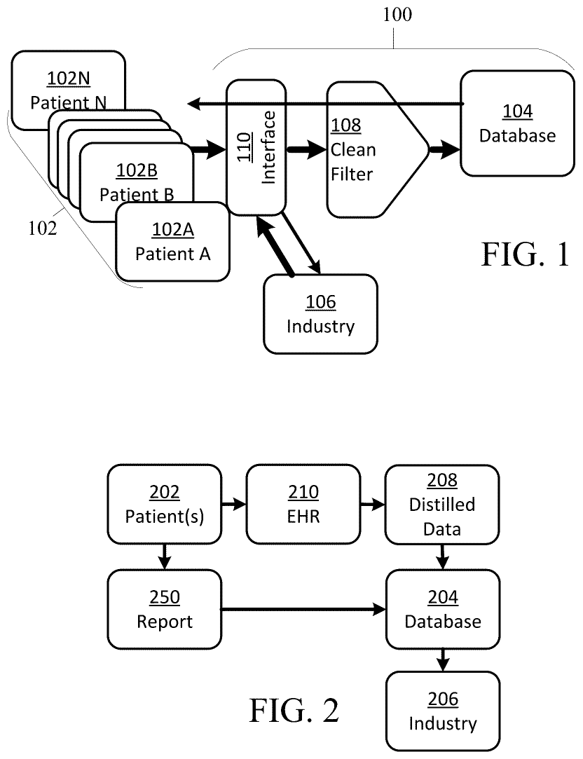

FIG. 1 illustrates an exemplary method diagram representing the ideas of the creation and use of the large-scale database described herein. As illustrated, the system 100 provides an interface 110 for any number of users 102 for receiving patient information to be stored in the database 104, that can then be used by the patients 102 or industry 106.

As illustrated, the system may permit the aggregation of any number of users 102. There may be a first user 102A, a second user 102B, up to an “Nth” patient 102N, where N could be any tens, hundreds, thousands, millions, or billions of patients.

System 100 is configured to have a user interface 110 to the one or more patients 102 to send and retrieve information from the one or more patients. The interface may be directly with the patient, may interface with one or more different electronic records system, may be with a doctor or healthcare provider, may be with an insurance company, etc. Accordingly, although the interface is illustrated between the patient and the database, the interface may be configured to communicate with any one or more source(s) of patient data as held by any one or more entities. The entered information 112 may be in digital form, may be a user entered information, or may be other forms of information.

Once entered into system 100, the entered information 114 may then be cleaned and/or filtered 108 as described herein. The cleaning and filtering may be to remove Personal Identifiable Information (PII) or otherwise remove, filter, rename, or modify the information to comply with limitations imposed by privacy concerns and/or filter the information to retain the most valuable (or other desired threshold of “desirable”) information to improve the database and/or perform any desirable analytics or processing as described herein.

Once the data is entered, filtered, and/or cleaned, the processed data 116 is then saved into the database 104. The patients 102 or industry 106 may then access and use the compiled data 118 as they need to to perform their own bio-computation.

Exemplary embodiments described herein may also permit the database 104 to receive additional information 122 into the system. For example, pharmaceutical companies may input information about drug information, such as, for example, recommended dosages, expected results, expected and/or possible side effects, etc. This information may be integrated into the database so that bio-computations can include different variables based on different associations.

Given the physical and nuanced nature of bio-computation, the analysis of exemplary databases described herein can be significantly augmented with physical analysis of samples derived from patients, which can be preserved and used in physiological microenvironments for testing that has exquisite predictive potential, as the supporting platforms enable systems that emulate patient pathology (patient-on-a-chip).

FIG. 2 illustrates an exemplary embodiment including bio-banking and its potential connection with bio-computation of pure information as shown and described with respect to FIG. 1. FIG. 2 illustrates an exemplary embodiment in which data is retrieved from patient 202 through an electronic health record (EHR) system 210 of the healthcare provider. Similar to FIG. 1 and other embodiments described herein, the received data may be filtered and/or cleaned to generate distilled data 208 that can be saved in the database 204. The system and method of FIG. 2 may also include obtaining biological samples 250 from the patient. The systems and methods may use the samples to generate additional information to store in the database and/or may bank the samples to be used as described herein. Information about the samples and/or derived from the samples (such as from additional testing), may be stored in database 204.

In an exemplary embodiment, during the diagnostic process, high-value samples 250 may be collected (either liquid or solid biopsy) from patient 202 and cryo-preserved. The patient retains the right to choose how their stored tissue is utilized. Information about the bio-sample 250 as well as directions about its use, such as, for example limitations of use, desired uses, etc., may be stored in database 204 along with the information received from the patient. This information can then be searched and/or used by industry 206 and retrieved from database 204.

For example, in a surgical intervention or when a patient has a biopsy, sufficient biological material may be collected for many tests, some may be run immediately to benefit the patient, others can be run later for the benefit of improved drug development or other industry applications. Additional information collected by third parties based on testing of micro-samples of patient tissue can help to combat the disease with greater precision, new drugs or combination therapies, or keep up with evolving disease, as often happens with recurrence of cancer.

In the preferred configuration, the patient-derived tissue is stored. The stored tissue can be tested by third parties as necessary or desired by the industry application. The patient retains the right to decide on tissue utilization. In one embodiment, the patient is spared the hassle of managing the tissue and a certain fraction of preserved biological samples are made available for research and development. The patient may provide use criteria or restrictions at the time of donation but thereafter is not needed for further authorizations or approvals to permit use of the biological sample.

Exemplary methods described herein include the principle and example details of how to create a novel database as well as non-obvious processes for dynamically populating and accessing it to enable medical queries while providing security and privacy of the patients. How the information is collected dynamically and assembled will be detailed further below.

Exemplary embodiments of the databases described herein enable the creation of massive amounts of human medical experiences from global populations. Such databases are difficult to create given the logistics of bringing together information and supporting such information to keep it up to date and useful. Handling such information at this scale is difficult to manage, maintain, and use as the scale causes inefficiencies and difficulties.

Exemplary embodiments of the systems and methods described herein include an expansive database (referred to herein as MDO). Exemplary embodiments described herein include medical data which has unique requirements. Exemplary embodiments described herein include the interconnection of small data systems into a larger collection.

The MDO has many contributors and users that can enlarge its size. Unlike most traditional physical resources, gold, coal, oil, and steel, digital collateral is not consumed with utilization. This MDO creates emergent value, which is a non-trivial result of the interaction of components it brings together.

Exemplary embodiments described herein join a large set of data that allows analytics that are impossible with fragments of medical data as currently exists. The conventional ecosystem of tiny data sets does not support large projects or analysis that can result in large benefits. The noise of medical and biological complexity does not allow the signal to be seen in a small dataset, while a very large dataset effectively reduces the noise allowing all kinds of signals or biological predictors to be identified.

Exemplary embodiments described herein may provide use that does not diminish the dataset, instead by creating additional information and links in the dataset, additional analytical insights may be made and used to guide the practice of precision medicine and derived revenue streams.

Exemplary embodiments herein may use the term bio-computation to cover the novel processes of data analysis. Select embodiments may use tissue to get biological information or (in silico) computation of biology and by biology, respectively. Bio-computation may include practical measurables of disease (biomarkers) and/or the ability to predict efficacious therapy.

Exemplary embodiments described herein include systems and methods of database systems able to accrue and fuse primordial and elementary data in a process. Exemplary embodiments may permit a critical mass of data to able continued use of the medical services, providing sustainable returns. Medical information may have significant sustainability and foster organic growth, which in the case of the systems and methods herein for data management comes from patents associated with diagnosis and treatment of diseases with precision and personalization of therapy.

Using exemplary embodiments of the systems and methods including the databases described herein permit patients to be able to tap into the experience of thousands of doctors and millions of patients to successfully predict personalized therapy. Biomarkers provide the foundation to identify effective therapy befitting an individual case. This is traditionally called precision medicine or personalized medicine that provides life-saving information such as “which therapy will save the life of a loved one?”.

Exemplary embodiments described herein generalize the search for critical healthcare information by introducing the term bio-computation. Bio-computation includes traditional bioinformatics, which is a form of computational biology, or more precisely computation of biology, with another concept of using patient's tissue, in computation by biology. The former is in silico calculations using biological measurables (biomarkers), the latter is in vitro testing of actual biology to reveal answers, where microscopic tissues, such as organoids are able to compute the results with the full complexity of the pathology, providing greater predictive value than any algorithm that is based on a limited set of input biomarkers. Exemplary embodiments of the physical bio-computation combine synergistically with the systems and methods including the databases described herein.

Exemplary embodiments described herein may integrate cloud-based systems and methods including the medical database described herein include services with use of bio-banking materials, such as a cryogenically frozen umbilical cord which is a source of embryonic stem cells, or biopsy tissue from a solid tumor. Briefly, the system and methods described herein enable us to set standards in physical measurements, which is desirable to transferability of data in the system.

In summary, exemplary embodiments described herein increase the power of biomarkers by analyzing them against a massive database of medical information delivered through cloud infrastructure or Software-as-a-Service (SaaS). A massive database with high quality precision medical data has the potential to transform the healthcare industry, empower the patient. As described elsewhere herein, exemplary embodiments described herein may provide passive income from revenue sharing, increasing efficiency of healthcare industry, and providing a foundation for digital collateral, with potential to rival and eventually exceed the financial value of payments for crude oil, the largest commodity that backs the world's reserve currency.

To assist in the maintenance and upkeep of the database, the system may be supported through financial incentives. For example, the medical data contained in the database can be very valuable and can serve as a source of revenue or digital collateral.

Exemplary embodiments described herein a process of monetization of medical data that rewards the patients, empowers pharmaceutical research and aligns interests of all healthcare providers.

Together financial innovation and bio-computation are able to generate new and emergent value and become self-sustaining and self-reinforcing. Exemplary embodiments described herein include connections between two separate branches of innovation to realize their potential for production of self-sustaining value. From the perspective of the total system (biotech+finance) the development of the systems and methods described herein may include an auto-catalytic process, where past a critical size, the database provides attraction for more data and users for continued sustainability.

Medical data has many interesting properties, including information with intrinsic value. Another remarkable property, which connects with the financial dimension of innovation is the inexhaustible nature of this resource. Every use of methods and processes described herein can be used to contribute to the database system described herein. There are approximately one trillion doctor's visits per year, indicating the potential for utilization of medical insights. The impact of the systems and methods described herein has implications beyond healthcare, including pure financial potential through the value of the data in the medical system and its continuous utilization.

The challenge of securing the value of this data can be accomplished by (i) creating a platform for storing Electronic Health Records (EHRs) focused on the patient and (ii) utilizing the system of associated de-identified searchable data. In the patient-focused EHR, which currently does not exist, exemplary embodiments are able to assemble copies of all medical records for the patient providing them with the benefits of comprehensive electronic records in a secure repository. Exemplary embodiments described herein may provide generous revenue sharing for the patients for sharing access to de-identified medical information in their EHRs. Systems and methods described herein may permit patients the rights to their medical data and can provide connection to comprehensive and continuously undated (living) medical records.

Exemplary embodiments described herein may provide additional information and value exceeding information typically available for clinical trials. As a point of comparison, monetary value of successful pivotal clinical trials can easily reach 2 million USD per patient. The systems and methods described herein can collect information that accumulates data equivalent to multiple clinical trials. Revenue sharing from monetization of the value of the information within the database far exceeds the nominal sell value.

Exemplary embodiments described herein include databases that may solve the problem of scale and fragmentation of medical markets. For example, there are niche markets for so-called orphan diseases, with small patient populations. In fact, one third of human diseases are rare diseases, making this situation quite common. Even in cases of common diseases, the nuances of individual health and pathology make the situations different, requiring several solutions or bins for classifying patients into different therapies. Attempts by companies to tackle this individually with small databases are extremely inefficient and can be compared to isolated data lakes versus a connected data ocean. Ever worse, presently the data does not flow, so instead of isolated data lakes, the information is more akin to isolated and frozen data lakes. The financial process that we describe below allows revenue sharing and alignment of interest of businesses with the patient resulting in a melt up of data. Melt up is a concept that is typically used in financial contexts to explain explosive growth and is the opposite of a meltdown. Creation of emergent financial values is part of the mutually reinforcing processes described herein.

Biotechnology and finance reinforce each other, providing a sustainable solution. Medical data also provides a uniquely scalable foundation for currency or digital collateral and an opportunity to store value, based on longevity and breadth of use of data. Unlike gold, investments in healthcare data provide continuous returns. That appreciation potential resulting from global use makes medical data uniquely suitable as a sound foundation for building prosperity at the level of a family, an investment fund, or a country.

Technical innovations are described herein that enable the systems and methods including the database (MDO), specifically regarding precision medicine data, and data reduction. These tools, including bio-computation, come from two categories—traditional bio-informatics tools, such as analysis of genomic mutations and the use of novel physical tools such as use of liquid biopsy to track cancer. Exemplary embodiments of bio-computation are described herein including processes of homogenizing, and integrating, and monetizing medical data.

Exemplary embodiments of the systems and methods including the medical database (MDO) are based upon shared data and precision medicine. Sharing medical data can create a true expanse of information by connecting existing data sets. Precision medicine (i) increases the personalization and therefore efficacy of therapy for the patient, (ii) connects medical information between patients to benefit from shared experience of others with similar pathology, and (iii) benefits drug development. One of the major requirements for Precision and Personalized Medicine (PPM) is availability of ample biomarker information. Biomarkers are biological measurements that can provide particularly relevant information for the disease. Examples include antibodies to COVID as well as mutations in oncogenes critical for progression of cancer.

Exemplary embodiments of medical systems and methods may use advanced multicellular models, especially those that simulate physiology of the disease. These Micro-Physiological Systems (MPS) can provide “direct drug response” information from what is aspiring to become a patient-in-a-dish model of disease. These microtissues are increasingly being stored in a process called bio-banking or cryogenic storage of viable tissue. Exemplary MPS platforms may complement bio-banking, which is currently seeing very limited usage, such as for long-term storage of umbilical cord tissue for its embryonic stem cells, although the applications of these cells for therapy are still in development. The applications currently available but with limited utilization include cancer therapy and neurodegenerative diseases. Future applications of MPS include tissue engineering to replace diseased or damaged organs. Exemplary embodiments described herein include disease applications of bio-banking in the greater context of bio-computation, which can also include autologous cell therapy.

Regarding analytical tools, exemplary embodiments described herein introduce standardization of reporting results of genomic sequencing for automated comparison and the corresponding meta-data. Briefly, Datum Distillation is a process of analysis of an individual Electronic Health Record (EHR) that results in de-identified and greatly reduced information with conclusion on biomarkers, including both measurable biologicals and keywords for symptoms, but no primary data. This process may use meta-data in primary files to the maximum extent. This includes the introduction of improved file management in the future physical processes (bio-computation). Specifically, exemplary embodiments associate information about not only acquisition parameters for the experiment but also references to control experiments files.

The homogenization process for data can be described as proper meta-data uses and the mathematical normalization process. Some instruments, such as magnetic resonance imaging (MRI) or computed axial tomography (CAT) scanners have effective factory calibration. In those cases, all that is necessary is to record a few details of the experiment, such as the contrast agent added, duration of the scan, and strength of the magnet. On the other hand, some instruments have significant calibration requirements including daily sensor calibration and controls for each experimental setup. For example, flow cytometry for at least four decades has been a golden standard in research analysis of cells in solutions, such as blood cells and especially white cells from the immune system, however, only recently did this analytical technique achieve regulatory approval for clinical applications. This has been due to lack of normalization and standardization techniques, specifically the intensity of the signal corresponding to each extracellular biomarker was reported in arbitrary units (AU), suitable only for internal comparison of cells. Lack of normalization made it impossible to compare experiments from different instruments and operators and time-points. The addition of simple calibration standards made it possible to normalize or convert the AU fluorescence to the actual number of protein receptors on the cell surface. This normalization accounts for the basic fluorescence calibration but does not take into the account details of the specific reagent including fluorescence (e.g. quantum efficiency given the number of fluorophores per antibody and length of the linker, photobleaching, etc.) or biological binding efficiency to the cognate receptor. While the food and drug administration (FDA) accepted a minimum threshold of normalization of a fluorophore conjugated to antibody, the agent of molecular recognition, there are additional sources of unaccounted variation, including the details of reagent and reaction set up. The extent of binding of antibody conjugate to the recognized antigen effects each experiment individually and is a known source of variation for various experiments, like immunohistochemistry and flow cytometry. The extent of the biological recognition reaction is very sensitive to variables that include time, temperature, agitation, and the quality of the antibody including activity that is a result of specific production lot and storage conditions.

To create a functional medical dataset (MDO), exemplary embodiments of the systems and methods described herein overcome the data transferability problem. Exemplary embodiments described herein index the control experiments and use normalization, as well as data markup. The markup introduces meta-data for each piece of information in the database and helps with the dynamic assembly of data sets in response to a medical query.

Exemplary embodiments of the medical dataset (MDO) described herein include precision medicine information. Exemplary embodiments of the medical dataset described herein may include biomarker measurements and response of the patient to therapy. A biomarker is a product of a specific use case application of diagnostic technology and patient selection, resulting in targeting a specific disease or a closely related family of pathologies. Measurement of a panel of critical biomarkers measures for a group of patients with the same disease can reveal the molecular fingerprints that identify the sub-population that responds to therapy.

Exemplary embodiments of the systems and methods described herein result in consistent and transferable measurements. This involves various control experiments and normalization calculations, to arrive at transferable units. For example, raw fluorescence data is reported in arbitrary units (AU) that are commonly used in scientific publications, however, are insufficient for clinical applications. The existing normalizations are both lacking in ability to account for enough sources of variation to ensure consistent measurements, as well as application of a variety of approaches. This results in heterogeneous methods without uniform standards, making assembly of different data sets very challenging (lack of data transferability). Creation of the medical dataset described herein has multiple benefits, including standardization across a very large dataset.

The other benefit of medical dataset (MDO) as shown and described herein is the impact on technical innovation. The precision medicine potential of the MDO relies on sophisticated diagnostic measurements being introduced into routine, standard use. Many innovative tools exist but are not being used for lack of regulatory approval, which stems from lack of clinical validation. There are many opportunities for technical innovation in the processes for effective and standardized utilization of these tools, as well as their integration technically and analytically. These opportunities involve collaboration with different end-users in academia, but especially industry; clinics and biotech companies, which ultimately falls in the category of biomarker. The creation of the MDO can spur adoption of existing cutting-edge tools and development of additional physical tools, in addition to the biomarker benefits described herein.

This disclosure includes descriptions and claims not only for technical innovation but also an infrastructure for transferring and sharing information. The infrastructure for sharing medical data, especially those that include clinical intervention, can help to align interests of pharmaceutical companies and insurance providers with those of patients. Exemplary embodiments of the systems and methods described herein accomplish the alignment and drive innovation by introducing a foundation for cryptocurrency, digital collateral, which is based on revenue sharing of a publicly traded company. To maintain the non-decreasing value of the digital currency, the supply is limited by a formula connected to the value of the data in the MDO with respect to revenue-generating potential.

This financial instrument can catalyze the improvements in the healthcare industry by enabling data sharing that is meant to reach a significant fraction of the global population. The vast use of medical information means significant financial potential for the MDO.

Exemplary embodiments of the system infrastructure for sharing medical information of the MDO include Digital Collateral (DiCo) derived from the MDO revenue. Revenue sharing can scale dramatically with use (DiCo-MDO).

Exemplary embodiments of the digital collateral (DiCo) described herein is ideally suited to the task of supporting cryptocurrency that would facilitate fundraising for a certain class of companies based around medical data analysis delivered through efficient SaaS platform providing a cost-effective way for innovators to connect with valuable clinical data.

Access to data can enable many new projects to create predictive algorithms. The biomarker information in the MDO includes analysis of all measurements from MRIs or immunohistochemistry slides to genomic data. Innovators who have cost-effective rented access to data in the MDO can contribute small data sets, novel scripts to analyze data and predict medical response. The value is captured based on datasets that pass clinical trial requirements focused on biomarker analysis for precision medicine.

A primary challenge of generating the medical dataset (MDO) as described herein is the transition from the current local optimum in healthcare to a global optimum. The current local optimum refers to the standard medical practices and associated financial processes, which can be described in function form measured by performance and cost, where financial incentives and the retention of data is on small local levels. However, getting to precision and outcomes-based medicine involves multiple parties changing behavior. Consensus can be driven with new financial structures. Without financial solutions, precision medicine technology alone is unable to drive the desired market penetration.

In addition to limitations on technical development, the clinical trial structure itself is limiting. Currently, clinical trials do not collect data continuously. Once a pivotal clinical Phase III trial meets its primary endpoints, typically based on a dataset of less than two thousand patients, there is no additional data collection and analysis. This lack of refinement of clinical practice is due to lack of financial incentive. Less than 3% of all interventional clinical trials in oncology are in Phase IV, the post-marketing follow up. This phase of clinical trials is about monitoring an approved drug regimen, however, there is typically no refinement of the application of the drug. The goal is merely to discover rare side effects, drug-drug interactions, and impact of diet that can impact the usage of the drug. A search for side effects is a far cry from continuous refinement, especially one that utilizes the precision and power of biomarkers.

More than new hardware and vast additional investments, the requirement for transition to the better state, which is characterized by a specific approach to medical practice and associated financial processes, involves creating new ways of sharing value (dividends from MDO revenue sharing) to align interests and change behavior(sic).

Patient experience offers valuable information and currently is not utilized to benefit other patients or drug developers with some rare and weak exceptions like Phase IV clinical trials. Conventional systems and methods are stuck in a local optimum characterized by lack of personalization and precision in medical treatment and high expenses. Sharing the sophisticated insights that can be gleamed from the treasure trove of medical data provides an unique opportunity to greatly enhance efficiency of healthcare domestically, as well as to create substantial revenue potential by selling access to the MDO globally.

Exemplary embodiments of the systems and methods herein, including the medical dataset (MDO), can enable drug developers to run more cost-effective projects.

The digital collateral associated with the sharing infrastructure described herein can empower fundraising for a new industry of companies that focus on precision medicine, delivered through SaaS to various existing and future hospitals and specialty clinics. Companies that build upon the foundation of the MDO will be using sophisticated analytical approaches, including artificial intelligence (AI), to develop biomarker combinations that provide clinically validated therapy options suited for each patient.

Exemplary embodiments described herein include systems and methods for medical data sharing including a Digital Collateral based on Medical Data (DiCo-MDO) that provides an option for revenue sharing equity. DiCo-MDO provides a unique foundation to other innovative financial tools, like cryptocurrency and Exchange Traded Funds (ETF). Medical data provides sustainable value, serving as a foundation for both the initial fundraising and the subsequent expansion phase of the new data-focused precision medicine companies.

Cryptocurrency generally brings three capabilities that facilitate initial fundraising, customer acquisition, and global expansion. In the past, access to sound financial foundation of digital collateral was missing. All the instruments such as Bitcoin or altcoins generally have relative, but not intrinsic value. This is especially the case of currencies such as Etherium and Solana, that facilitate development of other companies. Exemplary embodiments described herein of the systems and methods for medical exchanges create a system of medical data that in aggregate can be used as digital collateral, while protecting patients' privacy. The systems and methods described herein accomplish this through application of unique processes (Datum Distillation, Medical Data Fusion) that eliminate any personal identifiers or primary data, replacing it with quintessential biomarkers and keywords.

The original cryptocurrency Bitcoin is based on scarcity, while other so-called alt coins connect to various services that are tautological in being self-referential like discounts for trading (e.g. FTX) on a cryptocurrency exchange or the ability to get on their blockchain technology or its branch (e.g. ETH). Neither any cryptocurrencies nor Non-Fungible Tokens (NFTs), nor other digital assets that are representations of physical reality is suitable for liquid collateral. Medical data offers a unique opportunity to provide digital collateral with unambiguous value.

Exemplary embodiments of the systems and methods described herein for sharing medical information pool processed medical data to provide a source of clear value (better health outcomes, improved efficiency of drug development and healthcare system overall), yield of dividends through revenue sharing, and digital collateral for transactions. DiCo-MDO is flexible because it does not involve transfer of actual ownership of medical data, which always resides with the patient, but with sharing of revenue derived from its contribution to the MDO.

Exemplary embodiments described herein introduce digital collateral, which has multiple points of novelty and benefits. First, exemplary embodiments of the digital collateral described herein may function as a foundation for existing cryptocurrencies that can support scalable and sustainable revenue derived from DiCo-MDO. Second, it can serve as a foundation for other cryptocurrencies, including cryptocurrency options that can be converted to equity. It becomes a matter of preference for the customer to handle cryptocurrency or equity. Considering the value being created, exemplary embodiments of the digital collateral may be used to create other financial instruments, such as exchange traded funds (ETF) s that allow indirect investment in crypto.

An exemplary embodiment of a cryptocurrency option based on the systems and methods described herein can comprise very simple rules for the benefit of the users, namely that there is no cost of the option issuance and there is no time limitation for its conversion. The customer either uses the digital currency as a cash equivalent for short-and medium-term transactions as well as for its investment potential or they convert it to equity of the company holding the generating the currency, where longer term holding that financial instrument provides a dividend. After a certain point in corporate business development, such as an Initial Coin Offering (ICO), the quantity of the company is capped and is no longer created directly. After the ICO, the company shares can only be created by conversion of the digital currency, production of which is regulated by the value created by the company. Through such commitments and transparency, exemplary embodiments of the systems and methods can maintain fidelity with customers and patients.

Because the cryptocurrency (or digital currency) according to embodiments described herein, unlike equity in the company itself generating the currency, does not provide dividends or voting rights it is not equity, which is subject to the security and exchange commission (SEC) regulations. For all practical purposes, the digital currency, according to embodiments described herein, behaves like a cash equivalent, such as a cashier's check. The difference is that the cashier's check can be converted to cash at any bank, while the digital currency of the current disclosure can be converted to a common share of the company. At the time of the conversion, the equity needs to be deposited with an SEC-registered bank, trading platform or online exchange that has full compliance with Know Your Customer (KYC) and Anti-Money Laundering (AML) regulations. Company equity provides dividends distributed through the same regulations-compliant financial institution.

The MDO database contains medical data that is shared, enabling the system to maintain data ownership and privacy for the patient. Exemplary embodiments described herein may support auxiliary structure, an exchange that can rent data from hospitals and research institutions and offer it to industry, as well as trade financial derivatives based on medical data that is part of an industry we call Bio-Financial Innovation Technology (B-FIT). By monetizing data sharing, exemplary embodiments described herein can create and/or support a foundation of a new industry of algorithms and biomarkers that creates significant value. Creation of a new exchange could also manage crypto trading to maintain compliance and ensure the broadest possible application. In summary the MDO can provide the following:

Digital Collateral based on dividends from equity (DiCo-MDO) fosters investment in innovation through cryptocurrency. Cryptocurrency options provide a store of value while encouraging long term investments using existing stock exchanges to trade company equity, maintaining SEC compliance supporting creation of a new exchange data/financial trading platform.

New financial instruments derived from the MDO, DiCo-MDO and cryptocurrencies described herein facilitate the development of down-stream services that can leverage the MDO. The investment is self-reinforcing or auto catalytic. Young innovative companies can purchase access to the MDO using Galt and similarly, as they generate value-added analytical pre-dictions in specific pathology, can collect revenue through Galt or company/MDO revenue sharing. In this way, an exemplary subsidiary exchange can make revenue sharing agreements with companies that generate biomarker associations. These third-party companies use the MDO as a resource for developing biomarker combinations with strong clinical predictive potential as well as marketing the final product. The company generating the cryptocurrency can then accrue predictive power in different disease areas, while offering a single contact point for patients and doctors.

In an exemplary example, there is a portal facing patients and providers (hospitals and doctors) that provides medical information in a format suitable for each.

Exemplary embodiments of the digital currency (cryptocurrency) described herein may include different roles, functions, and/or objectives, including, in any combination and without limitation: to facilitate fundraising for a massive investment in healthcare technology and sharing; to provide digital collateral. In effect, the cryptocurrency described herein is equivalent to cash, suitable for settling transactions—potentially very large ones.

By providing security, privacy, and revenue sharing, the company behind the digital currency described herein can align interests of various parties in healthcare with those of the patient. Exemplary embodiments of the systems and methods described herein can be used to supply an SEC-registered cryptocurrency, which allows revenue sharing. The medical data accumulated collects both precision diagnostic data and patient response to therapy allowing massive data analysis, including using AI.

An example of an initial application of the digital currency infrastructure to facilitate the exchange of medical information is in the development of biomarkers for oncology. In this hypothetical situation a young company with specialty knowledge in a disease area, medical technology, or simply AI analytics focuses on a particular cancer and uses biomarkers to assign the most appropriate therapy option to individual patients. The cancer could be glioblastoma, at a specific stage with a specific history to resistance to therapy or relapses after a remission. The innovators use the MDO to select patients with this disease and patterns of biomarkers to identify effective therapies of different groups of patients. These algorithms use biomarkers to classify patients as belonging to specific cohorts, characterized by the most effective therapy. In this way the algorithms can predict very effective drug combinations for each patient. This analysis or study is considered retrospective by the FDA, which requires prospective verification with active enrollment for regulatory approval. However, even prior to the regulatory recognition this information can be lifesaving and can be sold to the patient and provider. Nevertheless, the path to full monetization of the biomarker information and its associations is impeded as the innovator has to trail-blaze the regulatory approval process. A massive MDO with quality analytics can provide strong predictive potential for therapy. Patients treated under this protocol follow minimal standard procedures of measurements of biomarkers and follow-up in open label distributed clinical trial. The MDO allows users to use other studies as “arms” of a common clinical trial, which develop independently. These arms can involve Standard-Of-Care (SOC) therapies, which make very helpful baseline comparison, especially for SOC in combination with a new agent.

The standardization that comes with the MDO also allows users to easily format and submit the data to regulatory agencies, including web interface with selective permissions to access data, and verify methodology. By working with exemplary embodiments of the systems and platforms described herein that use the same web-based tools, users can develop build-in validation of the processes used to analyze the data. Exemplary embodiments of the systems and methods described herein may combine standardized ways of storing data in the MDO with rich meta-data annotation and processes in place to track the transformations of data through all stages of analysis and final reporting to general a novel tool that can accelerate approval with the FDA.

An example is provided herein to illustrate the benefits of MDO for the innovator company, which only develops biomarker associations and works exclusively through a cloud portal. First, the user can leverage exemplary embodiments of the MDO to create a unique cryptocurrency tied to revenue sharing from personalized glioblastoma therapy to an initial fundraising. Their algorithms predict combinations of several off-patent drugs with complementary mechanisms of actions, validated with patient experience in retrospective analysis and then prospective enrollment in distributed clinical trials carried out by doctors globally under protocols with shared minimal requirements.

Exemplary embodiments of the shared medical database described herein are ideal for rapid accumulation of data in distributed clinical trials, addressing one of the major problems in the field—patient enrollment. Once the critical set of data is accumulated, the system and methods described herein can help with continuous communication with the FDA in a partnership process for collaborative acceleration of submission process whereby the system may preemptively address any concerns and ensure a rapid and positive response to the submission. Once biomarker associations are granted, the therapy option predicted by the algorithm can be submitted to insurance for reimbursement. This opens the business opportunity for the therapy option to be used by any patient globally who fits the biomarker profile. Doctors and hospitals greatly prefer the simplicity of administering approved therapy, such as based on the validated prediction delivered conveniently through a SaaS portal. Biomarker associations obtained by the innovator serves as a foundation for revenue sharing and connection with the system.

Access to the MDO delivered through the cloud can be creatively priced. In addition to cash equivalents, data can be purchased using cryptocurrency or through revenue sharing arrangements. Thus, the MDO provides a foundation of new industry with reduced overhead and business risk structure, which again supports the cryptocurrency investment potential while also providing the business owners with a choice of financial instruments to balance between fundraising needs and long-term revenue sharing.

FIG. 4 summarizes the multi-parametric optimization that supports the infrastructure of the medical data sharing as described herein. Exemplary embodiments of the systems and methods described herein for medical data sharing lie in the synergy of different fields: data, technology, and finance. Specifically, the change vector points from the current state, described on the three axes as the origin (0,0,0) to a global optimum of data sharing with generalized coordinates (D,T,F) that characterizes the advances along the three dimensions. First, exemplary embodiments described herein permit and facilitate the acquire a large amount of data (D) by handling it in a way that respects the patients' rights and empowers them. Second, exemplary embodiments described herein empower collection of precision medicine data using the latest technology (T), including systematic genomic analysis and liquid biopsy. Third, is the innovation in finance (F) that not only helps to raise funds to collect data and advance technology that helps to create MDO, but also leverages the tremendous potential for revenue sharing and can dramatically increase the efficiency in healthcare by aligning interests and empowering the analytical precision medicine industry that enables us to create value through biomarker associations.

Specifically, the creation of a massive precision medicine database can enable creation of algorithms that connect patients' biomarkers to the most successful therapy options. The increased measurement of biomarkers not only results in better health outcomes for patients but generates recurring value through MDO revenue sharing.

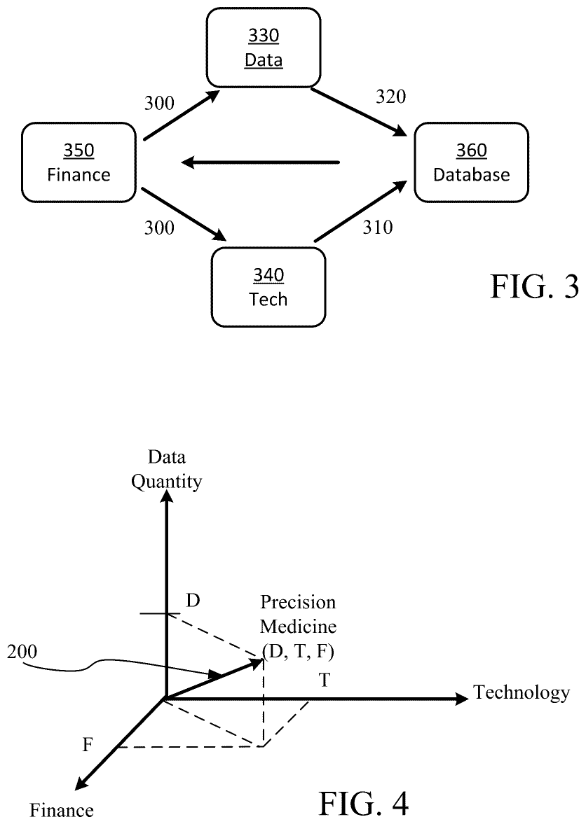

FIG. 3 shows the elements of creating and supporting the sharing of medical information. Specifically, financial innovations, including cryptocurrency options revenue sharing, etc. may be used to finance the building of the medical database (MDO) that involves the acquisition of lots of data, including measurements from novel and relatively expensive precision medicine technologies. MDO of a critical size and quality can generate revenue, closing the feedback loop with finance.

In an exemplary embodiment, a platform may be provided that may be accessed by users as a Software-as-a-Service (SaaS). For example, the system may be accessed through a web application that communicates with a server or through a local application that communicates with a remote server. The system may be configured to obtain data 330 from various sources and transmit the data 320 and store the data in a database 360. Additional information may be added to the database 360 through transmission 310 from precision medicine technologies 340. As the information from the database is access, the system may use cryptocurrencies 350 to pay for the receipt of the data 300. Exemplary embodiments may therefore permit financing 300 of data acquisition and health technology for data 330 and tech 340 contributions to the medical database that can then support revenue sharing and digital collateral from the MDO.

Exemplary embodiments described herein interconnect the roles of the three contributing factors illustrated in FIG. 4: finance, technology, data.

As we describe further in more detail, data sharing requires the ability to connect different types of information or similar information acquired differently. For example, cancer patients with the same disease may have different oncogene panels measured by using different providers. Measurements of certain biomarkers, such as proteins and cells can be challenging because different instruments can utilize different technologies. The normalization of these results requires adequate controls to account for nuances of this process of bio-computation, from the reagents and pre-analytical setup (extent of physical mixing, reaction time, temperature, buffer, etc.) to the sensors and analysis.

Exemplary embodiments of the systems and methods described herein empowers bio-computation, which in general means the acquisition of critical medical information, including in silico and in vitro methods. The former is the traditional sequence of diagnostics and bioinformatics, the latter means of using small pieces of tissue as an experimental computing entity. Exemplary embodiments described herein may use Micro-Physiological Testing (MPT) as a source of improved information for precision medicine and a critical innovation of bio-computation.

FIG. 2 shows the essence of monetization of medical data in a democratic and patient-centric paradigm: patient 202 contributes data, such as from patient medical records 210, that undergoes a distillation process 208, so that no medical identification information can be associated with the record. In an exemplary embodiment, the record may, at the patient's election, include an ID, which allows de-masking with the patient's permission. For example, when a patient chooses to enable alerts about findings relevant to his condition or treatment. Also, or alternatively in the distillation process 208, the mass of information may be reduced to essentials, hence the term distillation. Only the key concepts are key, including keywords from medical records and their associations and measurements of biomarkers, such as measures of intensity and distribution of a contrast agent in an MRI. Only the essential information is extracted for analysis, not the whole image. The distillation process not only speeds analysis, but standardizes the data enabling the system to mix different sources of data, including different hardware instruments, providers (hospitals), sources of reagents, etc. The system then stores the distilled data into the medical database 204, that can then be used by industry 206 according to embodiments described herein.

Exemplary embodiments may also permit the obtaining and retention of patient samples 250. Samples may be stored at physical facilities and indexed to the data within the database. The system may therefore permit physical testing against specimens based on the use of the industry 206.

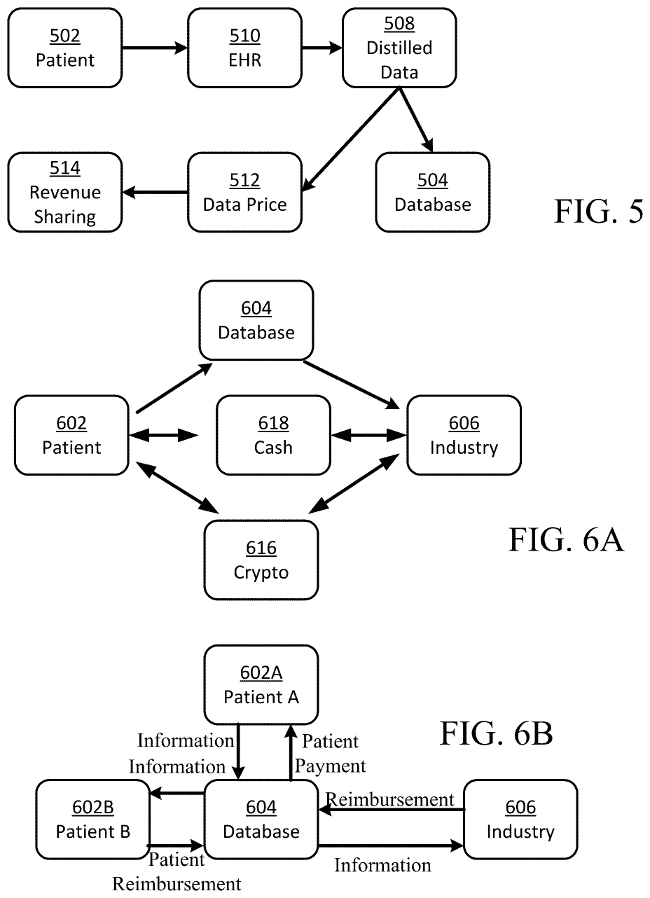

FIG. 5 also shows how exemplary embodiments of the systems and methods described herein manage the relationship with the patient, whose information exists in an Electronic Health Record (EHR). The patient 502 sees a provider or has medical information generated about them (such as through tests or other treatments), which is generally stored in an electronic health record 510 that is first distilled to generate distilled data 508. The database 504 is constructed primarily from contributions from “living” data—where a patient connects their EHR provider with the database and sets permissions for data sharing. The data in the EHR is priced 512 and revenue sharing 514 is provided back to the patient. The data price may be set according to the data's value as well as the scope and breadth of data sharing the patient selects. For example, the patient makes their data available for all academic, government, and industrial applications for tracking pathology and optimizing personalization and effectiveness of therapy. This helps the largest number of people and provides the greatest value that can be shared with the patient.

FIG. 6 illustrates an exemplary flow diagram of information in exemplary illustrations of the bio-computation database that connects different patients 602 with industry 606. Specifically, an exemplary specific precision therapy option, which is one equipped with diagnostic biomarkers, is used as an example of patient data entering the system during the clinical validation and after the resulting regulatory approval. At first, Patient A 602A that represents the initial “training set” contributes information to the database. These patients evaluate precision medicine options, and their primary mechanism of reimbursement is through the value of the data they share with the database, based on the Data Price. Data Price is set by the system for the patient's information with the goal of annual reimbursement. In the preferred configuration, revenue sharing is simplified and handled as cash 618 accrued to an account, that could be handled by PayPal or a similar service. Exemplary embodiments of the system may also or alternatively use re-investment, where a patient collects reimbursement in cryptocurrency 616 instead of cash. The default option is cash, because this gives the user the most simplicity and confidence in their reimbursement. They do not have to master cryptocurrency transactions or manage crypto investments to take full economic advantage of our invention.

In the exemplary embodiment, the flow of funds and value may be reversed, resulting in industry 606 compensating patients 602 for their valuable “living data”. Because these data are constantly updated, the records include information about patient medical records before therapy and after therapy, tracking recovery. This information helps to analyze patterns of response based on measurements of molecular biomarkers of disease, such as genomic and multi-omics analysis of other indicators of disease including specific proteins, metabolites, or multi-cellular organization that can be analyzed in a biopsy. It not only aligns interests of multiple parties (pharmaceutical and other companies, doctors, healthcare providers) with the needs of the patient, but motivates the patient to get organized with electronic medical records, to obtain the best diagnostic information and personalized therapy and to track response. The dual incentive of improved health and economic compensation creates a powerful driving force for the growth of the database. Once the database of high-quality data (that contains enough information about response to therapy and biomarkers that can be correlated with success) reaches a critical size, with enough patients in a specific disease category, it becomes possible to rent access to the database with Distilled Data to industry for virtual drug screening (VDS). VDS is a purely analytical technique to use patterns of biological response to a new therapeutic to relate it to dozens if not hundreds of existing drugs, thereby predicting some features of biological response. Exemplary embodiments may then be used by other patients, such as Patient B 602B to generate more information to be used from and to the database 604.

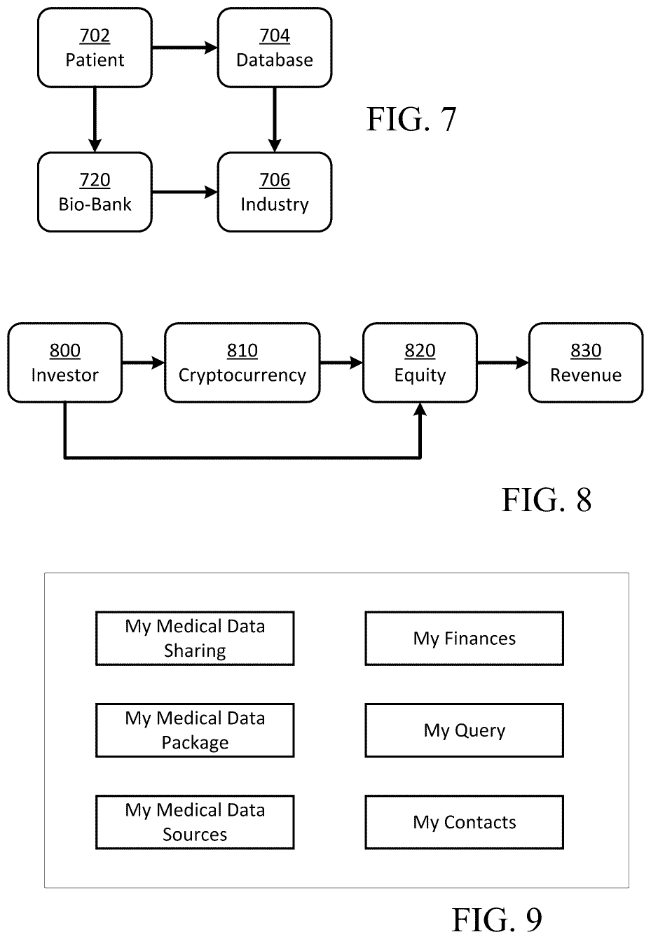

In fact, our database provides even more tools for the industry. FIG. 7 illustrates an exemplary embodiment of the role of bio-banking in bio-computation. The search for the right information in silico or using purely computational tools is based on patterns of previous measurements for existing drugs. The complex biological system that needs to be analyzed and fixed means that analysis alone is limited, even when based on large databases of high-quality measurements, and analyzed with the best algorithms, including artificial intelligence (AI). Certain forms of “computation” can result from testing bio-banking samples. For example, cancer biopsy samples can be used to produce multi-cellular biological constructs that include stem cells and are able to recapitulate many of the properties of the disease tissue. In other words, they provide a physiologically relevant model. Some of these models can be used for research and pre-clinical development, but there are clinical applications that are relevant to our process of bio-computation. An example of the latter is the use of organoids in tests that directly predict response to therapy options by testing them on the tissue. This process is called micro-physiological testing (MPT). So, two forms of bio-computation can be provided through exemplary embodiments of the systems and methods described herein: virtual drug screening (VDS) and/or MPT. Both processes can be searched for the same information, “which therapy will save the life of the patient?” can be considered forms of bio-computation. The MPT processes are more expensive and laborious but can be more comprehensive than analytical models. By physically considering the multiple pieces of information (e.g. biomarkers) the cells are able to provide a more realistic synthesis of complex information, such as dozens of genomic mutations or effects that can only observed in systems (such as a functional metabolizing organ). By providing MPT services, exemplary embodiments of the systems and methods described herein increase the value of VDS and empower personalized medicine for the donors and unprecedented bio-computation tools for the pharmaceutical industry.

FIG. 7 illustrates an exemplary flow diagram in which information is generated from a patient 702 and stored in the medical database 704 according to exemplary embodiments described herein. As described herein, the patient information may first be distilled before being stored. Patient samples may also be taken and stored in a biobank 720. The database may store information about the samples that can be used for further testing and the generation of information that can also be stored in the database. Industry may then use the database for data mining such as for virtual screening, or for finding relevant physical samples. Industry 706 may then use samples from the biobank 720 to generate more information relevant to particular testing and store additional information in the database 704.

FIG. 8 illustrates an exemplary process for bio-financial innovation, including an independent investor. These investors could include doctors, hospitals, and patients involved in precision medicine, but also venture capitalist and common investors. In fact, our preferred mechanism for investment includes cryptocurrency. This crypto can be traded by investors 800 on an exchange 810 freely and is similar to cash. Blockchain use enables tracking transactions on the exchange 810 and can be used to minimize any nefarious use. Cryptocurrency according to embodiments described herein can be exchanged for equity 820 in the company. Effectively, this cryptocurrency functions as a stock option enabling purchase of equity 820. Equity in a (publicly-traded) company can be used to provide a reliable and transparent mechanism of revenue sharing 830. Critically, this takes care of the gap in the capabilities of cryptocurrency—the anonymous nature of the accounts does not (yet) enable verification of customer identity. Specifically, anti-money laundering laws require equity traders to know-your-customer (KYC). The frequency of revenue sharing can be optimized to encourage investment. As illustrated, an investor 800 can purchase the cryptocurrency on the exchange 810. The investor can convert the cryptocurrency to equity 820 and obtain revenue from normal revenue sources, such as distributions, from the equity 820 in the company. As illustrated, there may be options for an investor to obtain equity 820 directly.

Thus, cryptocurrency and equity, together provide compliance with existing regulations and enable hand-off from one to the other. The price between the cryptocurrency and equity can be maintained through trades that establish an effective arbitrage system. In this way, the price of the cryptocurrency can reflect the yield generated by the equity in the company. Although equity in the company can be purchased directly some investors may prefer only to use the cryptocurrency for fast traders, looking for capital appreciation not yield.

FIG. 9 shows exemplary elements of the patient portal according to embodiments described herein. Exemplary embodiments of the patient portal described herein may provide a constant medical advisor available to each patient. Not only may this cloud-based SaaS “doctor” always be on call, but the patient may have the benefit of experience of thousands of doctors and millions of patients.

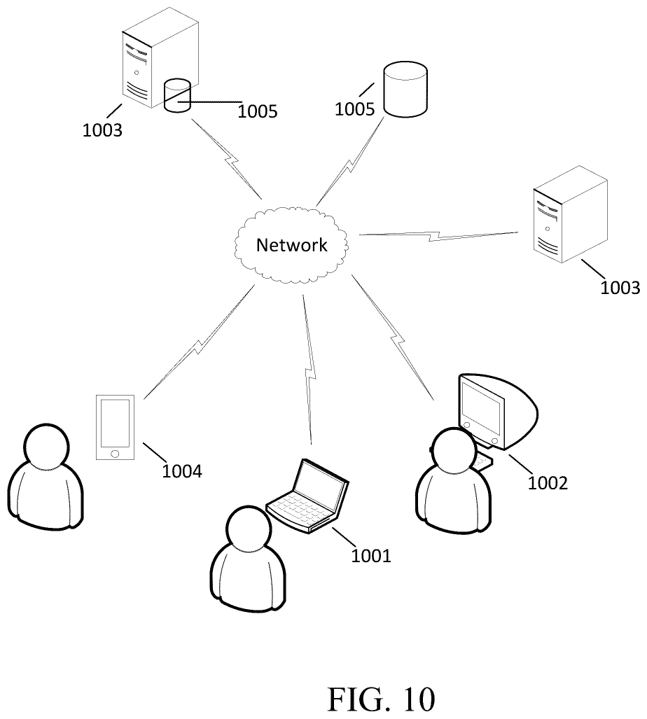

FIG. 10 illustrates an exemplary system used in the methods for sharing medical information as shown and described herein that can include the medical database. Exemplary embodiments of the system described herein may include a computer, computers, electronic device, or electronic devices. As used herein, the term computer(s) and/or electronic device(s) are intended to be broadly interpreted to include a variety of systems and devices including personal computers 1002, laptop computers 1002, mainframe computers, servers 1003, mobile phone 1004, tablet, smart watch, smart displays, televisions, and the like. A computer can include, for example, processors, memory components for storing data (e.g., read only memory (ROM) and/or random-access memory (RAM), other storage devices, various input/output communication devices and/or modules for network interface capabilities, etc.

In an exemplary embodiment, the system may include a processing unit including a memory, a processor, and a plurality of software routines that may be stored as non-transitory, machine-readable instruction on the memory and executed by the processor to perform the processes described herein. The processing unit may be based on a variety of commercially available platforms such as a personal computer, a workstation a laptop, a tablet, a mobile electronic device, or may be based on a custom platform that uses application-specific integrated circuits (ASICs) and other custom circuitry to carry out the processes described herein. Exemplary systems according to embodiments described herein include a processor and memory for storing and executing the routines according to embodiments described herein that are stored on servers and memory remote from a user. A user may then access the system through their own electronic device, such as a laptop or mobile phone. The user on their own device communicates with the remote server to send and receive information from the server through a network, such as the internet. The user's device may communicate with the remote server through a browser or other application running on the user's device.

Additionally, the user's device may use one or more input/output (I/O) devices that enable a user to interface to the system. By way of example only, the processing unit may receive user inputs through the user's device through the network to the server via a keyboard, touchscreen, mouse, scanner, button, or any other data input device and may provide graphical displays to the user via a display unit, which may be, for example, a conventional video monitor or touch screen. The system may also include one or more large area networks, and/or local networks for communicating data from one or more different components of the system. The one or more electronic devices may therefore input a user interface for displaying information to a user and/or one or more input devices for receiving information from a user. The system may receive and/or display the information after communication to or from a remote server 1003 or database 1005.

FIG. 9 illustrates exemplary processes and routines that may be performed by the system according to embodiments described herein. The different routines may be executed by a processor to provide displays to the user on a display screen to provide information to the user and/or receive information from the user. The different routines may be provided to different users as their needs require. Exemplary embodiments of the systems and methods described herein may use any combination of the routines described herein.

In an exemplary embodiment, the system may be configured with a registration routine that permits users to identify a user and log into the system. The system may permit users to enter identifying information such as name, contract information (address, email, phone number), system registration information (login and password), along with other information. The system may permit users to enter financial information. For example, the user may provide access to an account, such as a bank account or cryptocurrency wallet in order to make payments or receive payments into the system. The user may also set preferences about access to their information and/or to receive information from the system from the information provided or supplied to the system.

In an exemplary embodiment, the system may be configured with a user interface routine. The user interface routine may be configured to display a user interface to the user. The user interface routine may work with the other routines to achieve the objectives and functions of those routines by displaying or receiving information from the user.

The user interface routine may be configured to display a login screen to the user and receive login credentials from the user to permit the user to log into the system.

The user interface routine may be configured to provide a selection menu to a user and receive user inputs about the actions the user wishes to take. For example, the user may provide information to the system and/or search the system for information. The user interface routine may be configured to navigate to different pages for different routines or functions of the system and/or may open pop ups or other windows to provide and receive information to and from the user.

The system may include a Medical Data Sharing routine in which a user may set the preferences for the scope of data sharing. This could involve convenience for the patient as well as utility for research and development that ultimately provide revenue sharing.

Referring to FIG. 9, exemplary types of medical data that may be shared include any combination of” selective information or an entire datum of the user. Information may be shared between any user including, for example, between doctors to facilitate sharing data with a specialist; research institutions such as for academia and/or clinical research; insurance to check for competitive drug pricing or coverage; pharmaceutical companies to aid in drug development or drug prescription for a specific condition.

The system may include a Data Entry routine that permits users to enter in their own user information. The system may therefore permit the user to upload medical data files as well as record their own notes and observations. In effect, this portal becomes a user-friendly electronic lab notebook (ELN) for the patient to record their observations and to share them with their medical providers or to benefit other patients.

Referring to FIG. 9, the system may value personal and/or medical data. For example, the system may provide any combination of information for a user, such as, table of procedures and valuations, diagnostics, patient's notes, doctor's notes, response to therapy. Exemplary embodiments may use the information in different ways including sharing and/or compensating the user. For example, information may be shared to further develop the system and may compensate the user for use in sales and/or development applications. Exemplary embodiments of the system described herein may use primary data for biomarker development.

The system may include a Medical Data Sources routine configured to connect to third party electronic health records (EHR) s systems or electronic repositories. For example, the system may permit a patient to enter information to access their EHR system and receive electronic patient information from their doctor. The Medical Data Sources routine may be configured to extract and/or receive desired information from the EHR and communicate with the other routines of the system and/or store data into the health database. This can be helpful for centralizing medical records for a patient and for a family. Patients have the right to medical information in the format that is used by the provider. Increasingly, providers store information electronically, in which case it becomes a matter of managing permissions and security to obtain a copy of the records in a centralized repository, our B2C-focused EHR.

Referring to FIG. 9, the medical data sources, may include, for example, electronic health records, insurance records, pharmacy records, and/or other repositories of medical data.

The system includes a Medical Decision Assist routine for facilitating and/or providing relevant information and/or suggestions to a doctor or provider based on the health information of a patient that is supplied to the system. The Medical Decision Assist routine may be configured to facilitate medical decision making by doctors by connecting the medical experience of their patients to experience of many more similar patients. For example, the system may receive information about a patient and their condition and compare the information against other patient information and/or pathologies, symptoms, treatments, outcomes, side effects, etc. to provide additional insight to the doctor or providing in making further treatment decisions.

The system may include a Data Pricing routine and may provide a price schedule. For example, different pieces of information may be assigned a value. The system may present information to the user, such as, for example, in a table of entries including diagnostics, doctors' visits and patients observations with the corresponding price value. This panel provides transparency to the patient and motivates them to upload various medical data files as well as doctors' notes and to record their own observations. The Data Pricing routine may be configured to set prices according to the needs of the system. For example, if users have indicated a type of information, then the value for that information may increase. As another example, if data in a certain area is sparse, such as if patients routinely provide initial diagnostic information but not continued diagnostic or progression information, then the system may increase the value for the follow up information in the system to encourage participation. The open exchange and marketplace for information may also be used to value the medical data being entered. Any combination of data pricing may be used in any combination to value the medical data within the MDO and used by the system.

The system may include a Finances routine to provide basic tools to manage revenue sharing, selections between cash default options or cryptocurrency that can be converted to equity according to exemplary embodiments described herein. Other features include tracking reimbursement history, and possibly some medical expenses.

Exemplary embodiments of the system described herein may allocate and/or monitor financial compensation. Referring to FIG. 9, the my finances of the application may permit a user to be compensated in a cash or cryptocurrency option according to the embodiments described herein. The system may also include a history of payments. The system may also permit a user to connect to a bank or other financial institution or digital wallet to obtain the financial incentives and/or may purchase according to embodiments described herein.

The system may include a Query routine for the patient to ask medical questions and/or for users to search for specific information, such as conditions, pathologies, biomarkers, treatments, results, side effects, etc. from the medical information stored in the MDO and/or analyzed by the system. The query routine may allow the patient to select their medical record, such as in any combination of their existing observations, biomarkers, diagnosis, or treatment, to more easily pose a question that connects their concerns or evidence to experience of others with disease or conditions. The query routine may provide results of the query in different ways, such as, for example, summary of patient treatments corresponding to different search criteria, efficacy to treatment, comparison of treatments and/or results, statistics of percentage of participation of patients for each treatment and/or result, biomarkers or other characteristics relevant to the outcome of a treatment, etc.

Referring to FIG. 9, the query feature may permit a user to enter a medical query and obtain results from the mass of medical data of the system. Exemplary embodiments permit a user to search by symptoms, diagnosis, doctors, therapies, etc. Exemplary embodiments may permit a user to identify diagnostic options.

The system may include a Contacts routine configured to allow the patient to set preferences to being contacted. This can include basic cases like availability of new clinical trials that might interest the patient, new diagnostics or therapies, and/or sharing of patient information between different practitioners and/or relations (family or friends) of the patient. For example, the patient may select to share their information for use by industry to identify predictive biomarkers. Successful therapies in this category are inherently using precision medicine of biomarkers to select a new drug or a combination of drugs that is most fitting to the individual. When new actionable biomarkers are discovered, they are called companion diagnostics regarding the associated therapy. Therefore, the discovery of new interventional biomarkers is actionable and can also be of interest to patients looking outside of existing standard-of-care (SOC) therapy options. The patient can choose to be contacted in any or all of these cases involving recruitment into a new clinical trial(s), or development of new drugs or new interventional biomarkers.

Referring to FIG. 9, the contacts may permit the user to identify contacts. The contacts may be used to connect the user's information, such as by providing family, doctors, pharmacies, etc. together and related to a patient. The contacts may be used to connect the user or a contact of the user to third parties to complete one or more transactions. For example, a user may be connected to medical institutions or academia for medical trials. The user's doctors may be connected to institutions that can provide new diagnostic tools, new therapeutics, or combinations, or treatment options for the patient.

In an exemplary embodiment, the system is configured to set the medical value sharing (MVS) to approximately 4% of the data value. The data value can be priced according to embodiments described herein, such as using Data Package Pricing (DPP). Reimbursing patients based on a simple formula, such a percentage, adds confidence as does the use of cash as default reimbursement, instead of cryptocurrency. DPP can also be used to price virtual screening services, for example, by not only counting the number of patients grouped by pathology and possibly other factors, such as previous lines of therapy or current biomarkers, but also by adding the DPP value of the data. For example, a data package offered might include data from 20,000 patients, valued at 4 billion USD. Access to this data package can cost four million USD and would provide great savings to drug developers and enable projects which otherwise would be impossible due to funding requirements or challenges of enrollment. This data set is roughly ten times larger than data sets available for typical pivotal trials. Furthermore, the living or comprehensive nature of the data available from the EHRs provides access to medical information well before and well after treatment, making these data sets far superior to that information typically available in a clinical trial.

A pivotal Phase III clinical trial costs approximately 100,000 USD per patient and if successfully completed it is worth at least two million USD per patient. In other words, for the standard cost for 40 patients, it is possible to get access to information for 20,000. There is a nuance about retrospective and prospective enrollment, but that is easily resolved. The MDO can offer access to patients who have undergone specific therapy for retrospective analysis. This has the advantage of numbers—both the quantity of patients that can be analyzed as well as the number of years post-therapy. This follow up is desirable to measure statistical differences in effectiveness of various therapies for a specific condition. This retrospective analysis can allow innovators to identify the biomarker pattern associated with therapy success. The number of patients increases the statistical power of the predictive potential of the algorithm. However, the FDA only accepts clinical trials based on prospective enrollment. In other words, additional time would be needed to accrue patients who enrolled in the treatment arm of the clinical trial and undergo therapy predicted by statistically very well developed and capable algorithms. When the statistical results are replicated in the experience of a sufficient number of new patients, the clinical trial information can be submitted to the regulators.

In an exemplary embodiment, the system may include a Regulatory Submission routine that permits users to communicate with regulators in preparing for clinical trials or in providing submissions for drug information, for trials and/or post follow up. An exemplary benefit of the systems and methods described herein including the medical database (MDO) is the use of the combined information to facility communication with the regulators and improve drug and/or device approvals. Conventional communication with regulators is a major cost and hurdle for drug development.

Exemplary embodiments of the systems and methods described herein for medical sharing of information may include a medical database of information (MDO) that is accessible through a SaaS cloud-based platform that is ideally suited to communication with multiple participants including patients, providers, drug companies, medical device companies, testing facilities, and regulators.

The systems and methods for medical sharing of data can create a standard or pipeline for packaging data for regulatory submissions, such as 510(k). As described herein, exemplary systems and methods may include an associated exchange B-FIT that can be used with the access to the medical database (MDO) to manage trading of medical data and associated tools and financial instruments. Developers can use B-FIT to sell access to tools that take advantage of artificial intelligence (AI) to develop algorithms or technically facilitate image analysis or genomic analysis that can be applied to the data contained in the MDO. Similarly, researchers can pool resources to create better data sets contributing physical tools, analytical tools, access to patients among other contributions. B-FIT can help potential collaborators to find each other, to buy or sell access to data sets, and to help them market the results, including cases of incremental success. The ability to sell partial or incremental success is another benefit that may be realized with exemplary embodiments of the systems and methods described herein to reducing risks in the new bio-computation industry thereby facilitating fundraising using cryptocurrency.

Exemplary embodiments of the system and methods described herein may include a Data Distilling routine to filter and/or analyze the patient data before it is stored in the medical database. For example, the Data Distilling routine may de-identify the medical data to remove HIPAA identifiers and/or personally identifiable information that is protected and subject to sharing restrictions. The HIPAA privacy rule sets forth policies to protect all individually identifiable health information that is held or transmitted. Protected information can be used to identify, contact, or locate a single person or can be used with other sources to identify a single individual. When personally identifiable information is used in conjunction with one's physical or mental health or condition, health care, or one's payment for that health care, it becomes Protected Health Information (PHI).

In an exemplary embodiment, the Data Distilling routine may be configured to take the patient information, such as from the patient directly and/or through the electronic health records and de-identify the information before it is stored in the health database.

Two of the most important identifiers are photographic images that are limited to images of the face or any other characteristic that could uniquely identify the individual. The latter can include any primary personal health information (PHI) data.

Exemplary embodiments of the Data Distilling routine may be configured to remove this data from the MDO. The system, such as through the Data Distilling routine, may include an image analyzer that is configured to recognize identifiable features, such as eyes, faces, etc. and remove this information from the files before it is saved to the medical database. The Data Distilling routine may also or alternatively extract only the relevant information to the diagnosis, treatment, etc. and remove extraneous details to reduce the data set and/or reduce the likelihood of retaining personally identifiable information. The Data Distilling routine may therefore include image processing to identify target areas that are of interest and extract the relevant information related to the areas of interest and discard the remaining information from the image.

Exemplary embodiments of the Data Distilling routine may be configured to analyze the information received from the various data sources, such as from one or more of the other routines, such as, for example, the Medical Data Sources routine and/or Data Entry routine and transform the information into a unified data format. Exemplary embodiments may use uniform language for categorizing and/or describing the information, and/or may use a unified reference system such in quantities, efficiency standards, etc. Information may therefore be unified before it is stored in the medical database according to embodiments described herein.

Investment in medical healthcare provides great utility to the individual and society. Exemplary embodiments of the systems and methods described herein enable data joining and data sharing that allows re-utilization of medical investments and creates recurring value. Currently, data and experience of patients with the same disease are not broadly shared, resulting in inefficiency. The initial cost of collecting high-quality precision medicine data can make this life-saving information beyond the reach of many patients. Viewed as an investment it is possible to channel capital into generating this resource. The investors can take advantage of increasing value of medical datasets as it grows and becomes more sophisticated by effectively paying for advanced diagnostics. In return, investors collect cryptocurrency, which functions as cash associated with the dataset, and backed by the value of the data.

Exemplary embodiments of the systems and methods described herein may optionally include the bio-banking of patient samples. Exemplary embodiments of the system, such as in the data pricing routine and/or finances routine, patients may be compensated for the use of samples provided by the patient, such as the exemplary embodiment of from bio-banking embodiment of FIG. 2: The patient retains the rights to decide on tissue utilization and collects substantial monetary compensation, which is considerably higher than for data-only analysis, also known as virtual screening, where biological response to a new agent is related to patterns of response to existing drugs with known mechanisms of actions. In an exemplary optional embodiment, the patient may set preferences for the use of their samples and specimens so that the patient does not have to manage the tissue and a certain fraction of preserved biological samples are made available for research and development.

FIG. 1 illustrates an exemplary flow diagram of how exemplary routines and system components may be used in the systems and processes described herein.

For example, one or more patients 102 may interface with the system 100 through an interface 110 to provide information to the system and/or receive information from the system. Industry (other third parties) 106 may also provide information to the system and/or receive information from the system through another interface. The interface 110 for patients 102 and industry 106 may be the same interface or a different interface. The interface may be generated using the User Interface routine according to exemplary embodiments described herein.

For example, individual patients (identified as Patient A 102A, Patient B 102B, through Patient N 102N) can be any number of patients. The patients may supply information to the system 100 through the user interface 110 and/or through other routines as described herein, such as, for example, the medical Data Sharing routine, Data Entry routine, User Interface, and/or Registration routine. The received information may be cleaned 108, such as using the Data Distilling routine. The cleaned data may then be stored in the database 104.

In an exemplary embodiment, patients 102, industry 106, and/or other users may retrieve information from the system. For example, users may obtain assistance in medical diagnosis or other treatment planning using the Medical Decision Assist routine. Users may also or alternatively obtain information from the system using the Query routine. Different users, such as patients 102, industry 106, providers, and others may therefore access and retrieve information from the database 104.

In an exemplary embodiment, the transfer of the medical data may be accompanied by payment using the Data Pricing routine and/or Financial routine. Data from the database 104 may be packaged together to provide large quantities of information such as for industry 106 to identify correlations in biomarkers or other characteristics for diagnosis, treatment, and/or other information. Data going into the database, such as from patients, may be paid for at the time of sharing and/or at the time of use or purchase. For example, the data that goes into each purchased data set may identify a category of users that get paid whenever the dataset is purchased by industry. Additionally or alternatively, a patient may be paid for supplying their information to the database. The payment to the patient may be based on different characteristics. For example, the data may include a flat fee for inclusion into the database, the rate may depend on the level of use permitted by the user for the data, the rate may depend on the availability of a sample, the rate may depend on the amount of data (quantity and/or quality, duration, etc.), and any combination thereof or other considerations such as those described herein.

Compared with prior art, the present invention provides a comprehensive and integrated Medical Data Ocean (MDO) platform for consolidating medical data from various sources, while maintaining privacy and enabling monetization of the data for precision medicine applications. The present invention has the following beneficial effects:
1. Enables large-scale analytics on integrated medical data from disparate sources, allowing the identification of biological predictors and guiding the practice of precision medicine, which was previously hindered by fragmented data silos.
2. Fosters the growth of analytical insights and derived revenue streams from the MDO without diminishing the data itself, creating emergent value through the interaction of components brought together in the MDO.
3. Facilitates the joining of a large set of data, enabling analytics that are impossible with fragmented data lakes, as the noise of medical and biological complexity can be effectively reduced, allowing the identification of meaningful signals or biological predictors.
4. Utilizes biomarkers, such as antibodies to COVID and mutations in oncogenes, which are crucial for precision and personalized medicine (PPM), by providing ample biomarker information from the integrated MDO.
5. Incorporates micro-physiological systems (MPS) and bio-banking, enabling the storage and analysis of viable tissue models that simulate disease physiology, providing direct drug response information and supporting applications like cancer therapy and tissue engineering.
6. Employs datum distillation, a process of analyzing individual electronic health records (EHRs) to de-identify and greatly reduce information while retaining biomarkers, symptoms, and metadata, ensuring privacy and efficient data management.
7. Enables the monetization of the MDO data by creating artificial “farms” that grow analytical insights, fostering the development of precision medicine and derived revenue streams without consuming or diminishing the data itself.

The purpose of the invention is to overcome the drawbacks in the existing technology and provide a method for creating a Medical Data Ocean (MDO), which includes: Step 1, integrating medical data from disparate sources into a centralized MDO; Step 2, performing datum distillation on the integrated medical data; Step 3, judge if there are any new medical data sources or updates, if yes, return to Step 1, if no, proceed to Step 4; and Step 4, enabling monetization of the MDO data.

Exemplary embodiments of step 1 may include: (a) collecting medical data from various sources, including electronic health records (EHRs), biomarkers (e.g., antibodies to COVID, mutations in oncogenes), and micro-physiological systems (MPS) that simulate disease physiology; (b) standardizing and homogenizing the collected medical data into a common format for integration; (c) storing the integrated medical data in a centralized MDO platform, ensuring secure data transfer and compliance with privacy regulations.

Exemplary embodiments of step 2 may include: (a) analyzing individual EHRs to extract relevant information, such as biomarkers, symptoms, and metadata; (b) de-identifying the extracted information by removing personally identifiable information (PII) while retaining biomarkers and metadata; (c) reducing the extracted information to a distilled form, significantly decreasing the data size while preserving the essential elements.

Exemplary embodiments of step 4 may include: (a) enabling large-scale analytics on the integrated and distilled MDO data to identify biological predictors and guide precision medicine; (b) fostering the growth of analytical insights and derived revenue streams from the MDO without diminishing the data itself; (c) creating artificial “farms” that grow analytical insights from the MDO, enabling the development of precision medicine and derived revenue streams.

The technical means for creating the MDO includes integrating medical data from various sources, such as EHRs, biomarkers, and MPS. The integration process involves collecting data, standardizing and homogenizing it into a common format, and securely storing it in a centralized platform. The technical means also include performing datum distillation, which analyzes individual EHRs to extract relevant information, de-identifies the extracted data by removing personally identifiable information (PII) while retaining biomarkers and metadata and reduces the data to a distilled form for efficient storage and processing.

Additionally, the technical means incorporate advanced communication technologies and language translation services to cater to diverse patient populations, as well as machine learning and artificial intelligence techniques for advanced data analysis. The MDO platform also integrates other types of healthcare data beyond medical imaging, such as genomic sequencing results and metadata, to provide a comprehensive view of the patient's health status.

The technical means further include enabling large-scale analytics on the integrated and distilled MDO data to identify biological predictors and guide precision medicine. It fosters the growth of analytical insights and derives revenue streams from the MDO without diminishing the data itself, creating emergent value through the interaction of components brought together in the MDO. The technical means also involves creating artificial “farms” that grow analytical insights from the MDO, enabling the development of precision medicine and derived revenue streams.

Exemplary embodiments described herein include a method for creating a Medical Data Ocean (MDO) in the healthcare data management field, comprising the following steps: integrating medical data from disparate sources into a centralized MDO; collecting medical data from various sources, including electronic health records (EHRs) containing patient medical histories, biomarkers such as antibodies to COVID-19 and mutations in oncogenes critical for cancer progression, and data from micro-physiological systems (MPS) that simulate disease physiology, wherein the MPS data includes direct drug response information from patient-derived micro-tissue models; standardizing and homogenizing the collected medical data into a common format for integration, wherein the standardization process involves converting the data into a unified data structure and terminology, and the homogenization process involves removing inconsistencies and redundancies in the data; storing the integrated medical data in a centralized MDO platform, ensuring secure data transfer through encryption and compliance with privacy regulations such as HIPAA, wherein the MDO platform utilizes distributed storage and computing resources to handle the large volume of data; performing datum distillation on the integrated medical data; analyzing individual EHRs to extract relevant information, such as biomarkers, symptoms, and metadata, wherein the analysis is performed using natural language processing and machine learning techniques to identify relevant data from unstructured text and images; de-identifying the extracted information by removing personally identifiable information (PII) while retaining biomarkers and metadata, wherein the de-identification process involves techniques such as data masking, pseudonymization, and tokenization; reducing the extracted information to a distilled form, significantly decreasing the data size while preserving the essential elements, wherein the reduction process involves techniques such as data compression, feature selection, and dimensionality reduction, resulting in a distilled dataset with a size ranging from 10% to 30% of the original data; judging if there are any new medical data sources or updates, if yes, returning to the first 1, if no, proceeding to the next step; enabling monetization of the MDO data; enabling large-scale analytics on the integrated and distilled MDO data to identify biological predictors and guide precision medicine, wherein the analytics involve techniques such as machine learning, deep learning, and statistical modeling, and the identified predictors include biomarkers, genetic variants, and environmental factors associated with disease risk and treatment response; fostering the growth of analytical insights and derived revenue streams from the MDO without diminishing the data itself, wherein the analytical insights are used to develop personalized treatment plans, predictive models, and decision support systems, and the derived revenue streams include licensing of the analytical models, subscription-based access to the MDO, and partnerships with pharmaceutical companies for drug development; creating artificial “farms” that grow analytical insights from the MDO, enabling the development of precision medicine and derived revenue streams, wherein the “farms” are computational environments that simulate various scenarios and conditions using the MDO data to generate new insights and predictive models, and the derived revenue streams include licensing of the artificial “farm” environments and the insights generated therein.

Exemplary embodiments of the systems and methods described herein can include different inventions in which each may not be necessary for the operation of the entire system but instead may be selectively optional. For example, the technical set up of the digital collateral exchange and financial innovation underpinning this system can stand alone as a separate invention. As another example, the technical set up of the data collection and sharing of medical information can stand alone as a separate invention. Exemplary embodiments of the systems and methods described herein provide new products and novel processes that may be run independently and/or monetized separately.

Exemplary embodiments of the systems and methods described herein permit democratization of the process utilization of medical data. Patients own their data and can selectively share and monetize this information. Doctors and hospitals are incentivized to share data and collaborate, achieving better results, by realizing monetary compensation, and public recognition. The medical database (i) accrues legacy data and (ii) collects superior data based on modern diagnostics and (iii) allows selective access of patients, doctors, and biotechnology companies.

In summary, exemplary embodiments of the systems and methods described herein including the exchange of digital currency and/or collateral solution satisfies the regulatory requirements while achieving rapid growth and adoption of the platform as broadly as possible globally by leveraging cryptocurrency and associated Hyperledger technology, which is designed to provide security and to maintain track of access, which aligns perfectly with the monetization of a digital information asset.

Scalable and sustainable revenue sharing based on healthcare enables the creation of digital collateral that can impact innovation in healthcare and finance globally.

The system described herein may be used within a method for creating a Medical Data Ocean (MDO), including: receiving medical data from disparate sources; performing datum distillation on the received medical data and integration of the distilled medical data to form integrated and distilled medical data, wherein the datum distillation further includes standardizing and homogenizing the received medical data into a common format for integration and the standardization process involves converting the received medical data into a unified data structure and terminology, and the homogenization process involves removing inconsistencies and redundancies in the received medical data; storing the integrated and distilled medical data into a medical database, wherein the stored integrated and distilled medical data comprises a source for individual medical data within the integrated and distilled medical data; receiving a request for information from the large-scale analytics of the integrated and distilled medical data; analyzing the integrated and distilled medical data to identify biological predictors and guide precision medicine; using the analysis of the integrated and distilled medical data to develop personalized treatment plans, predictive models, and/or providing a response to the request for information; and tracking the source of individual medical data from the integrated and distilled medical data used to determine the response to the requested information and/or to develop personalized treatment plans.

The method's step of receiving medical data from disparate sources may further include collecting medical data from various sources, including individual electronic health records (EHRs) containing patient medical histories, biomarkers and mutations in oncogenes for cancer progression, and data from micro-physiological systems (MPS) that simulate disease physiology, wherein the MPS data includes direct drug response information from patient-derived micro-tissue models.

The distillation of the received medical data of the method may include converting the received medical data into numbers and keywords.

The distillation of the received medical data of the method may include analyzing individual EHRs to extract information, including biomarkers, symptoms, and metadata, wherein the analysis is performed using natural language processing and machine learning techniques to identify relevant data from unstructured text and images to generate the numbers and keywords.

The method may further include de-identifying the extracted information by removing personally identifiable information (PII) and retaining biomarkers and metadata, wherein the de-identification process involves any combination of deleting data, data masking, pseudonymization, and tokenization, wherein the de-identifying comprising retaining a source identifier for tracking the source of individual medical data.

The analytics of the method may involve techniques such as machine learning, deep learning, and statistical modeling, and the identified predictors include biomarkers, genetic variants, and environmental factors associated with disease risk and treatment response.

The developed personalized treatment plan for an individual generated by the method may include actionable medical insights for treating the individual based on presentation of biomarkers.

The method may further include presenting a first user interface on a first electronic device configured to a medical practitioner user and presenting a second user interface on a second electronic device configured to a patient user.

The second user interface presented during the execution of the method may be configured to present medical implications based on similarities in a medical history of the patient to other patients in the MDO.

The method may exclude sharing the source identifier as part of the response to the request for information.

The method may further include receiving the consent of a patient to receive information and providing new information to the patient based on a specific criterion related to the patient.

Datum distillation used during the method may include biomarker-style data extracted from an EHR by using automated language analysis (NLP) where keywords and their relationships are taken from a written description of the EHR, and where image data of MRI (magnetic resonance imaging), IHC (immunohistochemistry), and/or FISH (Fluorescent in situ hybridization) are reduced to a set of numbers related to critical observable data sets.

Data distillation of the received medical data from the method may include processing the received medical data to extract metrics comprising biomarkers, keywords, and numerical weights and measurements.

Data distillation of the received medical data from the method may include supplements the received data to provide estimates to fill data gaps and adjust the data to account for variations between sources of the received data using sparce matrices.

Data distillation of the received medical data from the method may include analytical processes to interpolate gaps in the biomarkers of the received medical data.

Analyzing the integrated and distilled medical data from the method may include using a digital twin and/or predictive algorithms that mimic medical systems.

Tracking the source of individual medical data of the method may include tokenization and an open ledger system.

The terminology used herein is for the purpose of describing particular embodiments only and is not intended to be limiting of the disclosure. As used herein, the singular forms “a,” “an” and “the” are intended to include the plural forms as well, unless the context clearly indicates otherwise. These terms are merely intended to distinguish one component from another component, and the terms do not limit the nature, sequence or order of the constituent components.

It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Throughout the specification, unless explicitly described to the contrary, the word “comprise” and variations such as “comprises” or “comprising” will be understood to imply the inclusion of stated elements but not the exclusion of any other elements.

In addition, the terms “unit”, “-er”, “-or”, and “module” described in the specification mean units for processing at least one function and operation and can be implemented by hardware components or software components and combinations thereof.

In this document, when terms such as “first” and “second” are used to modify a noun, such use is simply intended to distinguish one item from another and is not intended to require a sequential order unless specifically stated. In addition, terms of relative position such as “vertical” and “horizontal”, or “front” and “rear”, when used, are intended to be relative to each other and need not be absolute and only refer to one possible position of the device associated with those terms depending on the device's orientation.

An “electronic device” or a “computing device” refers to a device that includes a processor and memory. Each device may have its own processor and/or memory, or the processor and/or memory may be shared with other devices as in a virtual machine or container arrangement. The memory may contain or receive programming instructions that, when executed by the processor, cause the electronic device to perform one or more operations according to the programming instructions.

The terms “memory,” “memory device,” “computer-readable storage medium,” “data store,” “data storage facility” and the like each refer to a non-transitory device on which computer-readable data, programming instructions or both are stored. Except where specifically stated otherwise, the terms “memory,” “memory device,” “computer-readable storage medium,” “data store,” “data storage facility” and the like are intended to include single device embodiments, embodiments in which multiple memory devices together or collectively store a set of data or instructions, as well as individual sectors within such devices.

The terms “processor” and “processing device” refer to a hardware component of an electronic device that is configured to execute programming instructions. Except where specifically stated otherwise, the singular term “processor” or “processing device” is intended to include both single-processing device embodiments and embodiments in which multiple processing devices together or collectively perform a process.

The terms “instructions” and “programs” may be used interchangeably herein. The instructions may be stored in object code format for direct processing by the processor, or in any other computing device language, including scripts or collections of independent source code modules that are interpreted on demand or compiled in advance. Functions, methods, and routines of the instructions are explained in more detail below. The instructions may be any set of instructions to be executed directly (such as machine code) or indirectly (such as scripts) by the processor. For example, the instructions may be stored as computing device code on the computing device-readable medium.

The term “data” may be retrieved, stored or modified by processors in accordance with a set of instructions. For instance, although the claimed subject matter is not limited by any particular data structure, the data may be stored in computing device registers, in a relational database as a table having a plurality of different fields and records, XML documents or flat files. The data may also be formatted in any computing device-readable format.

The term “module” refers to a set of computer-readable programming instructions, as executed by a processor, that cause the processor to perform one or more specified function(s).

Although exemplary embodiments are described as using a plurality of units to perform the exemplary process, it is understood that the exemplary processes may also be performed by one or plurality of modules. Additionally, it is understood that the term controller/control unit refers to a hardware device that includes a memory and a processor and is specifically programmed to execute the processes described herein. The memory is configured to store the modules, and the processor is specifically configured to execute these modules to perform one or more processes that are described further below.

Further, the control logic of the present disclosure may be embodied as non-transitory computer readable media on a computer readable medium containing executable programming instructions executed by a processor, controller, or the like. Examples of computer readable media include, but are not limited to, ROM, RAM, compact disc (CD)-ROMs, magnetic tapes, floppy disks, flash drives, smart cards and optical data storage devices. The computer readable medium can also be distributed in network-coupled computer systems so that the computer readable media may be stored and executed in a distributed fashion such as, e.g., by a telematics server or a Controller Area Network (CAN).

Exemplary embodiments of the system described herein can be based in software and/or hardware. While some specific embodiments of the invention have been shown the invention is not to be limited to these embodiments. For example, most functions performed by electronic hardware components may be duplicated by software emulation. Thus, a software program written to accomplish those same functions may emulate the functionality of the hardware components in input-output circuitry. The invention is to be understood as not limited by the specific embodiments described herein, but only by scope of the appended claims.

As used herein, the terms “about,” “substantially,” or “approximately” for any numerical values, ranges, shapes, distances, relative relationships, etc. indicate a suitable dimensional tolerance that allows the part or collection of components to function for its intended purpose as described herein. Numerical ranges may also be provided herein. Unless otherwise indicated, each range is intended to include the endpoints, and any quantity within the provided range. Therefore, the range of 2-4, includes 2, 3, 4, and any subdivision between 2 and 4, such as 2.1, 2.01, and 2.001. The range also encompasses any combination of ranges, such that 2-4 includes 2-3 and 3-4.

Although embodiments of this invention have been fully described with reference to the accompanying drawings, it is to be noted that various changes and modifications will become apparent to those skilled in the art. Such changes and modifications are to be understood as being included within the scope of embodiments of this invention as defined by the appended claims. Specifically, exemplary components are described herein. Any combination of these components may be used in any combination. For example, any component, feature, step or part may be integrated, separated, sub-divided, removed, duplicated, added, or used in any combination and remain within the scope of the present disclosure. Embodiments are exemplary only, and provide an illustrative combination of features, but are not limited thereto.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be utilized for realizing the invention in diverse forms thereof.

1. A method for creating a Medical Data Ocean (MDO), comprising:
receiving medical data from disparate sources;
performing datum distillation on the received medical data and integration of the distilled medical data to form integrated and distilled medical data, wherein the datum distillation further includes standardizing and homogenizing the received medical data into a common format for integration and the standardization process involves converting the received medical data into a unified data structure and terminology, and the homogenization process involves removing inconsistencies and redundancies in the received medical data;
storing the integrated and distilled medical data into a medical database, wherein the stored integrated and distilled medical data comprises a source for individual medical data within the integrated and distilled medical data;
receiving a request for information from the large-scale analytics of the integrated and distilled medical data;
analyzing the integrated and distilled medical data to identify biological predictors and guide precision medicine;
using the analysis of the integrated and distilled medical data to develop personalized treatment plans, predictive models, and/or providing a response to the request for information; and
tracking the source of individual medical data from the integrated and distilled medical data used to determine the response to the requested information and/or to develop personalized treatment plans.

2. The method of claim 1, wherein receiving medical data from disparate sources further comprises: collecting medical data from various sources, including individual electronic health records (EHRs) containing patient medical histories, biomarkers and mutations in oncogenes for cancer progression, and data from micro-physiological systems (MPS) that simulate disease physiology, wherein the MPS data includes direct drug response information from patient-derived micro-tissue models.

3. The method of claim 2, wherein the distillation of the received medical data comprises converting the received medical data into numbers and keywords.

4. The method of claim 3, wherein the distillation of the received medical data comprises analyzing individual EHRs to extract information, including biomarkers, symptoms, and metadata, wherein the analysis is performed using natural language processing and machine learning techniques to identify relevant data from unstructured text and images to generate the numbers and keywords.

5. The method of claim 4, further comprising de-identifying the extracted information by removing personally identifiable information (PII) and retaining biomarkers and metadata, wherein the de-identification process involves any combination of deleting data, data masking, pseudonymization, and tokenization, wherein the de-identifying comprising retaining a source identifier for tracking the source of individual medical data.

6. The method of claim 5, further comprising not sharing the source identifier as part of the response to the request for information.

7. The method of claim 2, wherein the analytics involve techniques including any combination of machine learning, deep learning, and statistical modeling, and the identified predictors include biomarkers, genetic variants, and environmental factors associated with disease risk and treatment response.

8. The method of claim 1, wherein a developed personalized treatment plan for an individual includes actionable medical insights for treating the individual based on presentation of biomarkers.

9. The method of claim 1, further comprising presenting a first user interface on a first electronic device configured to a medical practitioner user and presenting a second user interface on a second electronic device configured to a patient user.

10. The method of claim 9, wherein the second user interface is configured to present medical implications based on similarities in a medical history of the patient to other patients in the MDO.

11. The method of claim 1, further comprising receiving a consent of a patient to receive information and providing new information to the patient based on a specific criteria related to the patient.

12. The method of claim 2, wherein datum distillation comprises retaining biomarker-style data extracted from an EHR by using automated language analysis (NLP) where keywords and their relationships are taken from a written description within the EHR, and where image data of MRI (magnetic resonance imaging), IHC (immunohistochemistry), and/or FISH (Fluorescent in situ hybridization) are reduced to a set of numbers related to observable data sets.

13. The method of claim 12, wherein the data distillation of the received medical data includes processing the received medical data to extract metrics comprising biomarkers, keywords, numerical weights, and measurements.

14. The method of claim 13, wherein the data distillation further comprises supplements the received data to provide estimates to fill data gaps and adjust the data to account for variations between sources of the received data using sparce matrices.

15. The method of claim 13, wherein the data distillation uses analytical processes to interpolate gaps in the biomarkers of the received medical data.

16. The method of claim 1, wherein analyzing the integrated and distilled medical data further comprises using a digital twin and/or predictive algorithms that mimic medical systems.

17. The method of claim 1, wherein tracking the source of individual medical data comprises tokenization and an open ledger system.
